(19) United States
(12) Patent Application Publication
(10) Pub. No.: US 2007/0232653 A1
(43) Pub. Date: Oct. 4, 2007

(54) Process for the preparation of enantiomerically enriched cyclic beta-aryl or heteroaryl carbocyclic acids

(76) Inventors: Stephan Bachmann, Allschwil (CH); Michelangelo Scalone, Birsfelden (CH); Patrick Schnider, Bottmingen (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/731,191

(22) Filed: Mar. 30, 2007

(30) Foreign Application Priority Data
Apr. 3, 2006 (EP) 06112171.1

(51) Int. Cl. A61K 31/445; A61K 31/382; A61K 31/192; C07D 211/32; C07D 335/02

(52) U.S. Cl. 514/317; 514/326; 514/568; 514/432; 546/225; 549/13; 562/492

(57) ABSTRACT

The present invention relates to a process for the preparation of cis substituted cyclic β-aryl or heteroaryl carboxylic acid derivatives in high diastereo- and enantioselectivity by enantioselective hydrogenation in accordance with the following scheme

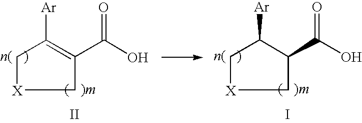

wherein
X, Ar, n, and m are defined herein and corresponding salts thereof.

### PRIORITY TO RELATED APPLICATIONS

This application claims the benefit of European Application No. 06112171.1, filed Apr. 3, 2006, which is hereby incorporated by reference in its entirety.

### BACKGROUND OF THE INVENTION

The synthesis of cis-substituted cyclic β-aryl or heteroaryl carboxylic acid derivates of general formula I from compounds of formula II is very poorly described in the literature. The reason seems to be the reluctance of the tetra substituted double bond of compounds of formula II to undergo the catalytic hydrogenation.

Synthesis of an enantiomerically pure acid of type I has been described in Bioorg. Med. Chem. Lett. 1998, 8, 2495 as shown in the following scheme:

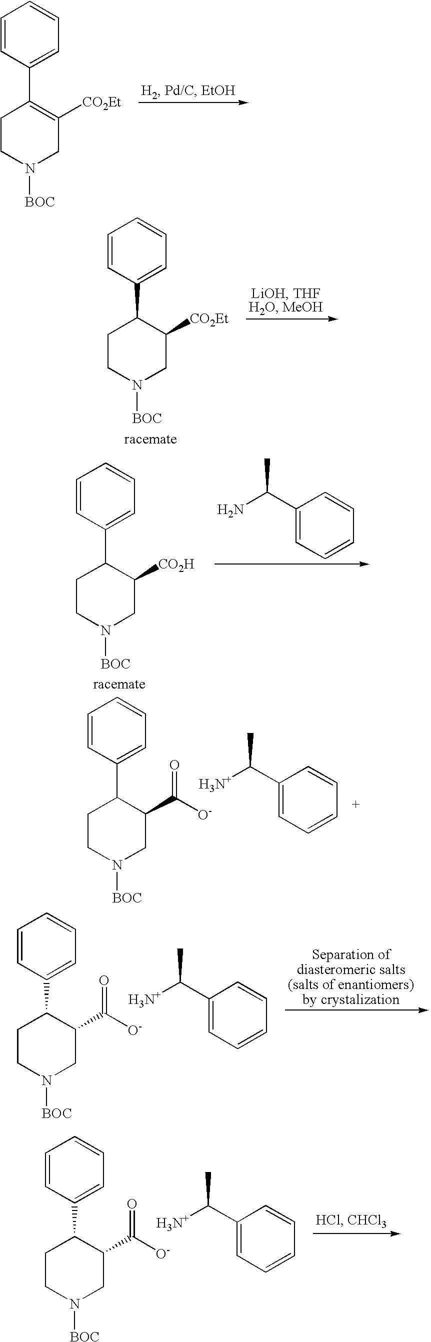

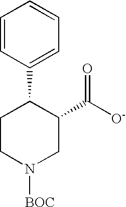

The synthetic pathway described suffers from three major drawbacks as compared to the present invention:

1) The ester derivative of an acid of type II was hydrogenated to the RACEMIC ester of I, which was consecutively saponified under carefully controlled conditions to the RACEMIC acid I. After salt formation with a chiral amine the diastereomeric salts could be separated by crystallization. The pure enantiomer was generated by treatment with acid.

2) Saponification of the RACEMIC cis-ester is problematic and may lead to partial epimerization to the trans-ester or acid, resulting in loss of material.

3) The method described in Bioorg. Med. Chem. Lett. 1998, 8, 2495 is not general: under the hydrogenation conditions using Pd/C aromatic groups such as indole or functional groups on the aromatic ring such as, e.g., nitro, chlorine, bromine or iodine substituents, which are sensitive to reduction, are usually not tolerated. Chlorine, bromine or iodine are usually replaced by hydrogen under such conditions.

Synthetic access to such compounds I is generally particularly difficult as the cis-substituted form is thermodynamically less stable than the trans form. Thus synthetic procedures under equilibrating conditions usually give rise to either cis/trans-mixtures or predominantly the trans form. In fact, selective epimerization of the chiral center α to the carboxyl group of enantiomerically enriched cis-substituted cyclic β-aryl or heteroaryl carboxcylic acid derivates I to the trans isomers IV is effectively done as follows:

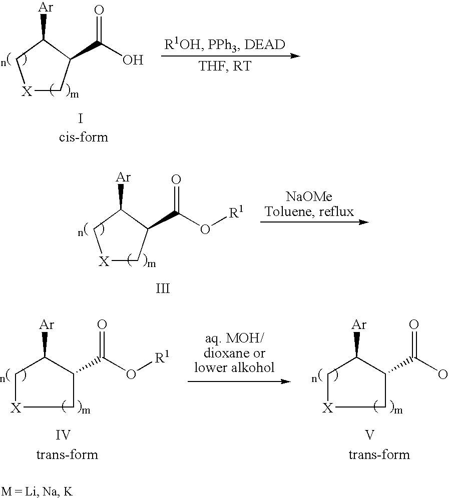

wherein R¹ is C₁₋₇-alkyl or benzyl, Ar is aryl¹ or heteroaryl¹.

Chiral enantiomerically enriched or pure compounds of formula I or V and their derivatives are of great interest, e.g., as intermediates or starting materials for the preparation of a range of pharmaceutically active compounds.

Fiduxosin (ABT-980), α₁ₐ-adrenoreceptor antagonist, a development compound at Abbot for the treatment of benign prostate hyperplasia, is disclosed in Organic Process Research & Development 2004, 8, 897-902 and references cited therein.

The synthetic route for preparation of Fiduxosin is as follows:

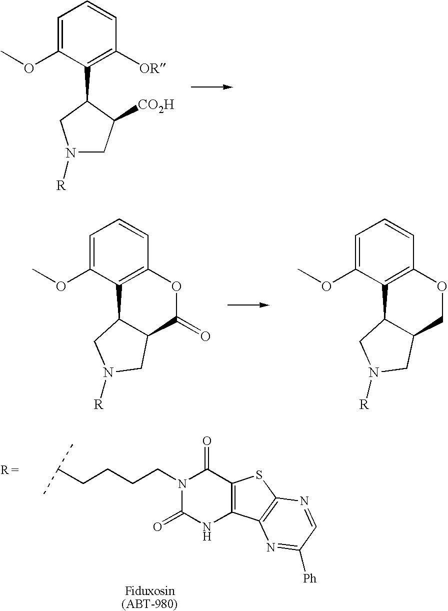

Melanocortin-4 receptor agonists are known for the treatment of obesity. They are described in Bioorg. Med. Chem. Lett. 2003, 13, 4431 & 2005, 15, 4023 (Merck Sharp & Dohme):

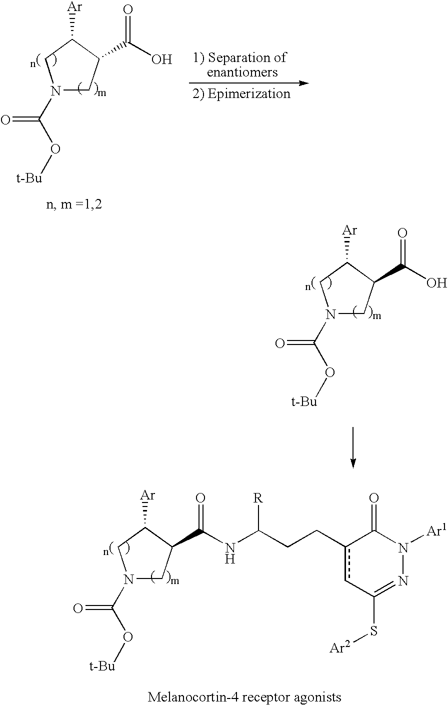

Melanocortin-4 receptor agonists are known for the treatment of obesity. They are described in WO02068388 and J. Org. Chem. 2005, 70, 3592 (Merck Sharp & Dohme):

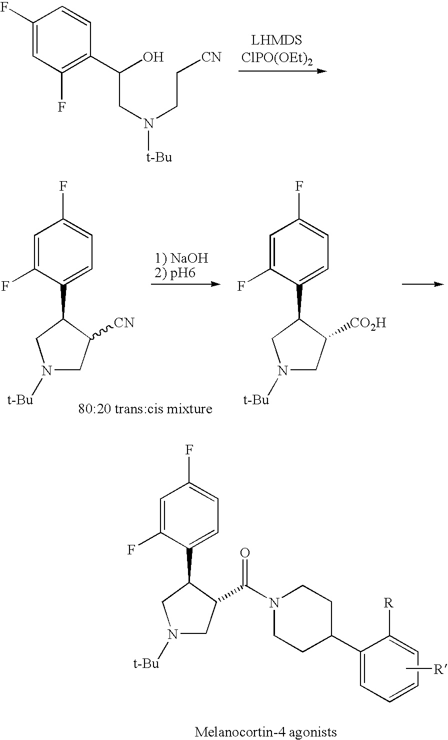

Chemokine receptor CCR5 antagonists are known for the treatment of viral infections. They are described in Bioorg. Med. Chem. Lett. 2004, 14, 941 (Merck Sharp & Dohme):

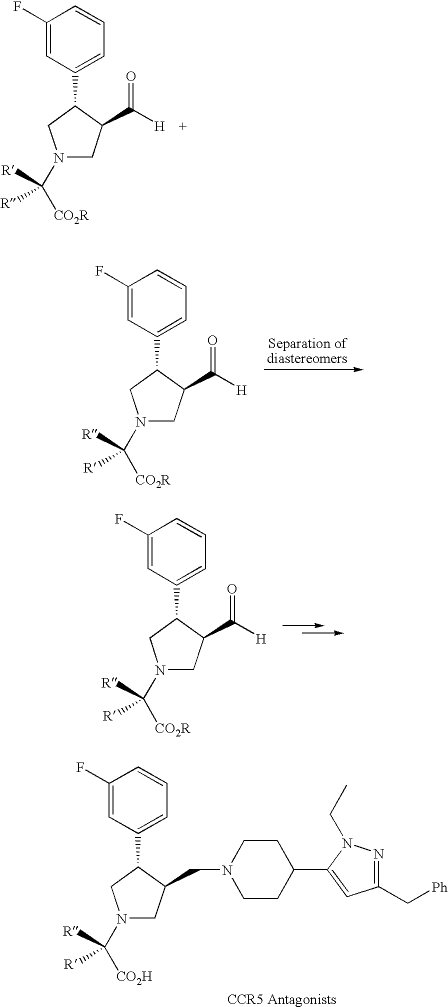

Chemokine receptor CCR5 antagonists are known for the treatment of viral infections. They are described in Bioorg. Med. Chem. Lett. 2001, 11, 1437 (Merck Sharp & Dohme):

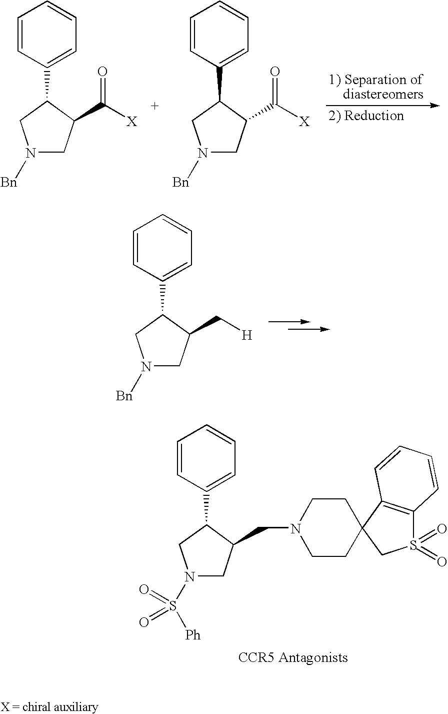

Factor Xa inhibitors are known as antithrombotic agents. They are described in Bioorg. Med. Chem. Lett. 1999, 9, 1195 (DuPont):

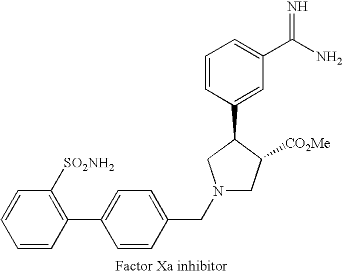

The selective serotonin reuptake inhibitor paroxetine is marketed for the treatment of depression and anxiety. The process for the preparation of paroxetine is described in WO0129031:

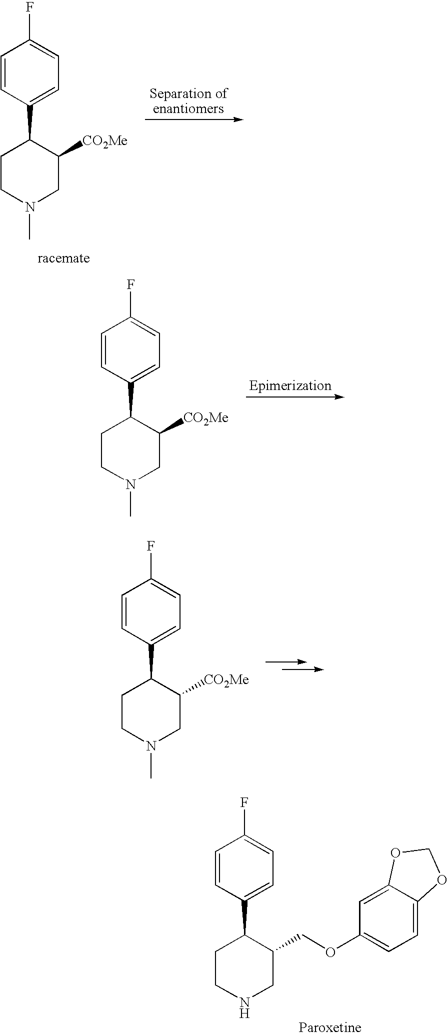

Dopamine and norepinephrine uptake inhibitor (+)—CPCA, potentially useful for the treatment of cocaine dependence and craving. is described in The Journal of Pharmacology and Experimental Therapeutics 2003, 305, 143:

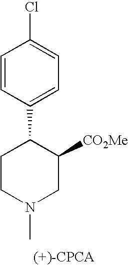

Tachykinin receptor antagonists, potentially useful preventives or remedies for lower urinary tract dysfunction, digestive organ diseases or central nervous diseases are disclosed in WO2005068427:

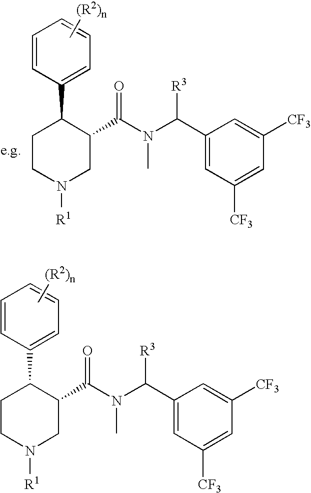

Dual Neurokinin-1 receptor antagonists and selective serotonin reuptake inhibitors, that are useful, e.g., for the treatment of depression and/or anxiety are described in US Patent Publication 2006/0020011:

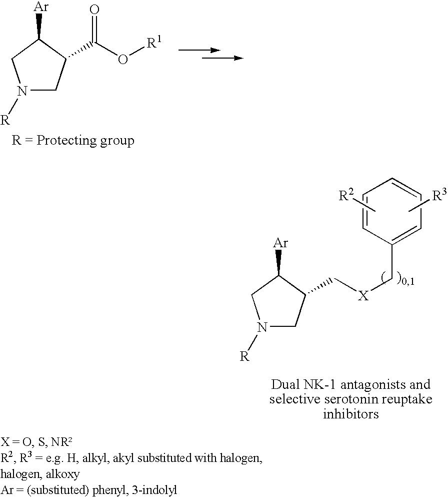

### SUMMARY OF THE INVENTION

The present invention provides a process for the preparation of cis substituted cyclic β-aryl or heteroaryl carboxylic acid derivatives in high diastereo- and enantioselectivity by enantioselective hydrogenation in accordance with the following scheme

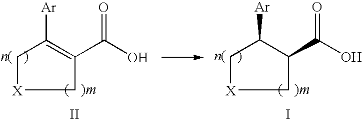

wherein
X is —C(R)(R′)—, —N(R″)—, —O—, —S(O)ₒ—, C(O)N(R″), —N(R″)C(O)— or —C(O)—;
R and R′ are each independently hydrogen, C₁₋₇-alkyl, C₁₋₇-alkyl substituted by halogen, C₁₋₇-alkoxy, hydroxy or —(CH₂)ₚ—Ar;
R″ is hydrogen, C₁₋₇-alkyl, C₁₋₇-alkyl substituted by halogen, —S(O)ₒ—C₁₋₇-alkyl, —S(O)ₒ—Ar, —S(O)ₙ—NRR′, —(CH₂)ₚ—Ar, —C(O)—C₁₋₇-alkyl, —C(O)—Ar, —C(O)—NRR′ or —C(O)O—C₁₋₇-alkyl;
Ar is aryl¹ or heteroaryl¹;
n is 0, 1, 2 or 3;
m is 0, 1, 2 or 3;
o is 0, 1 or 2;
p is 0, 1, or 2;
   and corresponding salts thereof.

The enantioselective hydrogenation of cyclic β-aryl or heteroaryl substituted α,β-unsaturated carboxylic acids II is the only method to give direct access to enantiomerically enriched cis-substituted cyclic β-arylcarboxyclic acid derivatives I.

The present invention also provides new compounds of formula I, which have been prepared by the above-mentioned process. These compounds include
(+)-(3R,4R)-4-(4-fluoro-phenyl)-piperidine-1,3-dicarboxylic acid-1-tert-butyl ester and
(−)-(3S,4S)-4-(4-fluoro-phenyl)-piperidine-1,3-dicarboxylic acid 1-tert-butyl ester,
(−)-4-(1H-indol-3-yl)-piperidine-1,3-dicarboxylic acid-1-tert-butyl ester and
(+)-4-(1H-indol-3-yl)-piperidine-1,3-dicarboxylic acid-1-tert-butyl ester,
(−)-4-o-tolyl-piperidine-1,3-dicarboxylic acid 1-tert-butyl ester and
(+)-4-o-tolyl-piperidine-1,3-dicarboxylic acid 1-tert-butyl ester,
(+)-4-(3-methoxy-phenyl)-piperidine-1,3-dicarboxylic acid-1-tert-butyl ester,
(+)-4-phenyl-piperidine-1,3-dicarboxylic acid-1-tert-butyl ester and
(−)-4-phenyl-piperidine-1,3-dicarboxylic acid-1-tert-butyl ester
(+)-3-phenyl-piperidine-1,4-dicarboxylic acid 1-tert-butyl ester and
(−)-3-phenyl-piperidine-1,4-dicarboxylic acid 1-tert-butyl ester,
(−)-2-phenyl-cyclopentenecarboxylic acid and
(+)-2-phenyl-cyclopentenecarboxylic acid,
(+)-(3R,4R)-4-(phenyl)-pyrrolidine-1,3-dicarboxylic acid-1-tert-butyl ester and
(−)-(3S,4S)-4-(phenyl)-pyrrolidine-1,3-dicarboxylic acid-1-tert-butyl ester,
(−)-4-(4-chloro-phenyl)-pyrrolidine-1,3-dicarboxylic acid-1-tert-butyl ester and
(+)-4-(4-chloro-phenyl)-pyrrolidine-1,3-dicarboxylic acid-1-tert-butyl ester,
(+)-4-(3-fluoro-phenyl)-pyrrolidine-1,3-dicarboxylic acid-1-tert-butyl ester and
(−)-4-(3-fluoro-phenyl)-pyrrolidine-1,3-dicarboxylic acid-1-tert-butyl ester,
(3R,4R)-1-benzyl-4-phenyl-pyrrolidine-3-carboxylic acid and
(3RS,4RS)-1-benzyl-4-phenyl-pyrrolidine-3-carboxylic acid,
(−)-2-phenyl-cyclooctanecarboxylic acid,
(+)-4-phenyl-tetrahydro-thiophene-3-carboxylic acid and
(−)-4-phenyl-tetrahydro-thiophene-3-carboxylic acid

The compounds of formula Ic be used as starting materials or intermediates for the preparation of pharmaceutically active compounds, especially for compounds, that are useful for the treatment of central nervous system disorders.

### DETAILED DESCRIPTION OF THE INVENTION

Unless otherwise indicated, the following definitions are set forth to illustrate and define the meaning and scope of the various terms used to describe the invention herein. The following definitions of the general terms used in the present description apply irrespective of whether the terms in question appear alone or in combination. It must be noted that, as used in the specification and the appended claims, the singular forms “a”, “an,” and “the” include plural forms unless the context clearly dictates otherwise.

The term “halogen” refers to fluorine, chlorine, bromine and iodine, with fluorine, bromine and chlorine being preferred.

The term “lower alkyl” or “C₁₋₇alkyl”, alone or in combination with other groups, refers to a branched or straight-chain monovalent hydrocarbon radical of one to seven carbon atoms, preferably one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like. Preferable lower alkyl residues are methyl and ethyl, with methyl being especially preferred.

The term “halogenated lower alkyl” or “C₁₋₇alkyl substituted by halogen” refers to a lower alkyl group as defined above wherein at least one of the hydrogens of the lower alkyl group is replaced by a halogen atom, preferably fluoro or chloro. Among the preferred halogenated lower alkyl groups are trifluoromethyl, difluoromethyl, fluoromethyl and chloromethyl.

The term “lower alkoxy” or “C₁₋₇alkoxy” refers to the group R′—O—, wherein R′ is lower alkyl as defined above. Examples of lower alkoxy groups are e.g. methoxy, ethoxy, propoxy, isopropoxy, butoxy, isobutoxy and hexyloxy, with methoxy being especially preferred.

The term “cycloalkyl” refers to a monovalent carbocyclic radical of three to eight, preferably four to six carbon atoms. This term is further exemplified by radicals such as cyclopropyl, cyclobutyl, cyclopentyl and cyclohexyl, with cyclopentyl and cyclohexyl being preferred. Such cycloalkyl residues may optionally be mono-, di- or tri-substituted, independently, by lower alkyl or by halogen.

The term “aryl” refers to an optionally substituted aromatic monovalent mono- or polycarbocyclic radical having 6 to 10 ring atoms.

The term “aryl¹” refers to an aromatic monovalent mono- or polycarbocyclic radical having 6 to 10 ring atoms-, such as phenyl or naphthyl, preferably phenyl, which can optionally be substituted by one or more substituent, independently selected from C₁₋₇-alkyl, hydroxy, C₁₋₇-alkoxy, —O—C₁₋₇-alkyl substituted by halogen, —O-benzyl, —OC(O)—C₁₋₇-alkyl, —OC(O)-phenyl, halogen, C₁₋₇-alkyl substituted by halogen, cyano, amino, mono- or di-C₁₋₇-alkyl amino, —NHC(O)—C₁₋₇-alkyl, —NHC(O)-phenyl, —S(O)ₒ-amino, —S(O)ₒ-mono- or di-C₁₋₇-alkyl amino, —S(O)ₙ—C₁₋₇-alkyl, —S(O)ₙ—C₁₋₇-alkyl substituted by halogen, nitro, —C(O)OH, —C(O)—O—C₁₋₇-alkyl, —C(O)—O—C₁₋₇-alkyl substituted by halogen, —C(O)—O-phenyl, —C(O)—C₁₋₇-alkyl, —C(O)—C₁₋₇-alkyl substituted by halogen, —C(O)-amino, —C(O)-mono- or di-C₁₋₇-alkyl amino, —C(O)—NH-phenyl or the like.

The term “heteroaryl¹” denotes a monovalent heterocyclic 5 or 6-membered aromatic radical having 5 to 12 ring members, wherein the ring heteroatoms are selected from N, O and S, for example the groups thiophenyl, indolyl, pyridinyl, pyrimidinyl, imidazolyl, piperidinyl, furanyl, pyrrolyl, isoxazolyl, pyrazolyl, pyrazinyl, benzo[1.3]dioxolyl, benzo{b}thiophenyl or benzotriazolyl. Heteroaryl¹ can optionally be substituted independently by one or more substituent, such as C₁₋₇-alkyl, hydroxy, C₁₋₇-alkoxy, —O—C₁₋₇-alkyl substituted by halogen, —O-benzyl, —OC(O)—C₁₋₇-alkyl, —OC(O)-phenyl, halogen, C₁₋₇-alkyl substituted by halogen, cyano, amino, mono- or di-C₁₋₇-alkyl amino, —NHC(O)—C₁₋₇-alkyl, —NHC(O)-phenyl, —S(O)ₒ-amino, —S(O)ₒ-mono- or di-C₁₋₇-alkyl amino, —S(O)ₒ—C₁₋₇-alkyl, —S(O)ₒ—C₁₋₇-alkyl substituted by halogen, nitro, —C(O)OH, —C(O)—O—C₁₋₇-alkyl, —C(O)—O—C₁₋₇-alkyl substituted by halogen, —C(O)—O-phenyl, —C(O)—C₁₋₇-alkyl, —C(O)—C₁₋₇-alkyl substituted by halogen, —C(O)-amino, —C(O)-mono- or di-C₁₋₇-alkyl amino, —C(O)—NH-phenyl or the like.

The term “aryl²” refers to an aromatic monovalent mono- or polycarbocyclic radical having 6 to 10 ring atoms, such as phenyl or naphthyl, preferably phenyl, which may optionally be independently substituted by one or more substituents, selected from C₁₋₇-alkyl, C₁₋₇-alkoxy, halogen, C₁₋₇-alkyl substituted by halogen, cyano, azido, amino, mono- or di-C₁₋₇-alkyl amino, SO₂H, SO₂-lower alkyl, nitro, C(O)O—C₁₋₇-alkyl, C(O)-mono- or di-C₁₋₇-alkyl amino, hydroxy or the like.

The term “heteroaryl²” denotes a monovalent heterocyclic 5 or 6-membered aromatic radical, wherein the heteroatoms are selected from N, O and S, for example the groups thiophenyl, indolyl, pyridinyl, pyrimidinyl, imidazolyl, piperidinyl, furanyl, pyrrolyl, isoxazolyl, pyrazolyl, pyrazinyl, benzo[1.3]dioxolyl, benzo{b}thiophenyl and benzotriazolyl. The heteroaryl optionally can be substituted by one or more substituents, independently by C₁₋₇-alkyl, C₁₋₇-alkoxy, halogen, C₁₋₇-alkyl substituted by halogen, cyano, azido, amino, mono- or di-C₁₋₇-alkyl amino, SO₂H, SO₂-lower alkyl, nitro, C(O)O—C₁₋₇-alkyl, C(O)-mono- or di-C₁₋₇-alkyl amino, hydroxy or the like.

The term “anionic ligand” denotes halides or the group A-COO⁻, wherein A represents lower alkyl, aryl², halogenated lower alkyl or halogenated aryl².

The term “neutral ligand” denotes e.g. olefins, e.g. ethylene, propylene, cyclooctene, 1,3-hexadiene, 1,5-hexadiene, norbornadiene (nbd=bicyclo-[2.2.1]hepta-2,5-diene), (Z,Z)-1,5-cyclooctadiene (cod) or other dienes which form readily soluble complexes with rhodium or ruthenium, arenes, e.g. benzene, hexamethylbenzene, 1,3,5-trimethylbenzene, p-cymene, or also solvents such as e.g. tetrahydrofuran, dimethylformamide, acetonitrile, benzonitrile, acetone, methanol and pyridine.

The term “chiral phosphine ligand” denotes a chiral organic compound containing at least one phosphorus atom capable to coordinate to a transition metal. Suitable tertiary monophosphines can have three O-bonded or one N-bonded and two O-bonded substituents.

The term “chiral diphosphine ligand” denotes a diphosphine in which the two tertiary phosphorus atoms are bound to different carbon atoms of linear or cyclic bridging groups, preferably bound to different carbon atoms of a carbon chain having from 2 to 6 carbon atoms, where the carbon chain may be part of a monocyclic ring or part of a bicyclic ring system (e.g. biphenyl, binaphthyl, bisthienyl or cyclopentadienylphenyl, cyclopentadienyl-CH2-phenyl, cyclopentadienyl-CH(OCH3)-phenyl in ferrocenes). Particularly, the chiral diphosphine ligand is a chiral ligand characterized by formula (3), (4), (5), (6), (7), (8), (9), (10), (11), (12), (13), (14), (15) or (16).

The term “non-coordinating anion” denotes anions such as a halide, BF₄⁻, ClO₄⁻, SbF₆⁻, PF₆⁻, B(phenyl)₄⁻, B(3,5-di-trifluoromethyl-phenyl)₄⁻, CF₃SO₃⁻, C₆H₅SO₃⁻.

The term “halide” denotes fluoride, chloride, bromide and iodide.

The process for homogeneous enantioselective hydrogenation as described in the present invention offers a viable method for the reduction of compounds of formula II to compounds of formula I. The reduction can be carried out much more economically than known processes, with less process steps, under moderate conditions, and with high yields. Further, crude intermediate products can mostly be used in subsequent reaction steps without the need of any additional purification steps.

Advantageously, the stereochemical integrity of the stereocenter β to the carboxylic function bearing the aryl or heteroaryl group is preserved during the epimerization. Therefore, enantioselective hydrogenation of acids II as described herein is unique in that it allows access to all possible stereoisomers of cyclic β-aryl or heteroaryl carboxylic acids and their derivatives in enantiomerically enriched or pure form, i.e. cis-substituted acids I and their derivatives as well as trans-substituted esters IV and acids V and their derivatives.

Homogeneous catalysts for this conversion should be active under relatively mild conditions, to allow the achievement of high diastereo- (i.e. cis/trans ratio) and enantio- (i.e. R,R/S,S ratio) selectivities. From highly enantiomerically enriched cis-configurated acids of formula I also the corresponding acids with trans-structure are easily accessible by epimerization of the center a to the carboxylic function.

In comparison with the direct enantioselective hydrogenation of the invention, known processes are tedious, require at least three additional steps and are not atom-economical as at least 50% of the material is lost during the separation of the enantiomers in the form of their diastereomeric salts.

The reaction conditions described in the present invention using a homogenous palladium complex are compatible with reducible groups, such as aromatic groups, e.g., indole or functional groups on the aromatic ring, such as, e.g., nitro, chlorine, bromine, or iodine substituents.

Synthetic access to the starting materials II for the enantioselective hydrogenation is straightforward from readily available β-ketoesters VII. A number of such compounds VII are commercially available. Compounds VII can also be prepared in a straightforward manner from ketones VI by consecutive treatment of a ketone VI with a suitable base, e.g. lithium diisopropylamide, lithium hexamethyldisilazide, alkyllithium with or without additives such as N,N,N′,N′-tetramethylethlenediamine, lithium, sodium or potassium alkoxide or sodium hydride in a suitable solvent such as tetrahydrofuran followed by a source of the carboxylate moiety, e.g. an alkyl or benzyl chloroformate or carbonate. β-Ketoesters VII can be transformed into triflates VIII by treatment with a bases such as sodium hydride and a triflating agent such as N-phenyltrifluoromethanesulfonimide. Coupling of a triflate VIII with an arylating agent such as, e.g., arylzinc halide or arylboronic acid or ester using a suitable palladium catalyst such as tetrakis(triphenylphosphine)palladium gives esters IX which are saponified in the usual manner to acids II.

### Synthesis of Starting Materials:

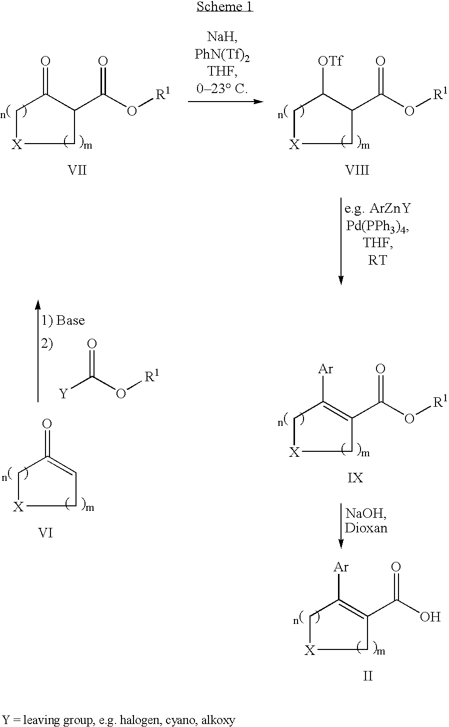

wherein R¹ is C₁₋₇-alkyl or benzyl, Ar is aryl¹ or heteroaryl¹, X is —C(R)(R′)—, —N(R″)—, —O—, —S(O)ₙ—, C(O)N(R″), —N(R″)C(O)— or —C(O)—; R and R′ are each independently hydrogen, C₁₋₇-alkyl, C₁₋₇-alkyl substituted by halogen, C₁₋₇-alkoxy, hydroxy or —(CH₂)ₚ—Ar; R″ is hydrogen, C₁₋₇-alkyl, C₁₋₇-alkyl substituted by halogen, S(O)ₙ—C₁₋₇-alkyl, S(O)ₒ—Ar, S(O)ₒ—NRR′, —(CH₂)ₚ—Ar, —C(O)—C₁₋₇-alkyl, —C(O)—Ar, —C(O)—NRR′ or —C(O)O—C₁₋₇-alkyl; n and m are independently from each other 0, 1, 2 or 3; o is 0, 1 or 2; and p is 0, 1, or 2.

As illustrated by the examples above, acids I and V, wherein X═NR″, are of particular interest as precursors for the synthesis of, e.g., pharmaceutically active ingredients. 1-Benzyl-3-oxo-piperidine-4-carboxylic acid ethyl ester and 1-benzyl-4-oxo-piperidine-3-carboxylic acid methyl and ethyl ester are commercially available and are thus the most convenient starting materials VII-1 for the synthesis of acids II, wherein X═NR″ and n=1 and m=2 or n=2 and m=1. For practical reasons it may be advantageous to change the N-protecting group from benzyl to tert-butoxycarbonyl (BOC), e.g. as described in scheme 2.

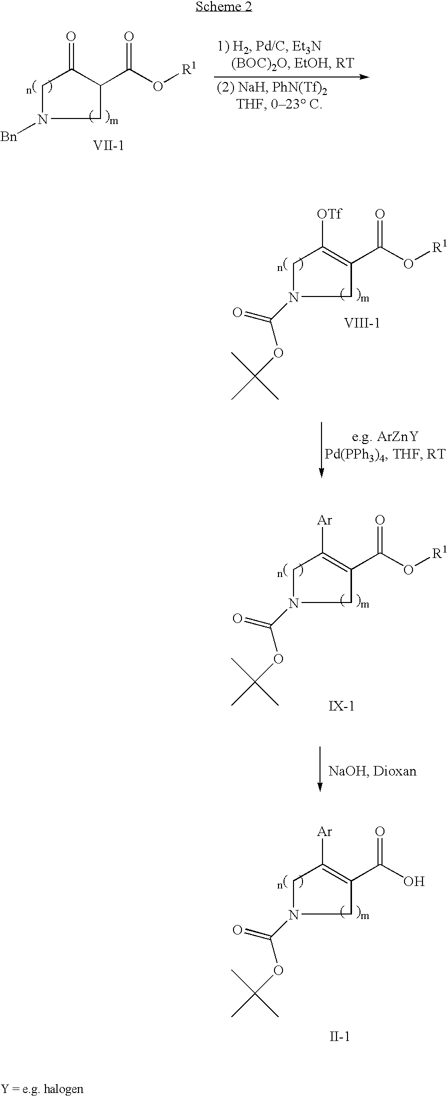

wherein R¹ is C₁₋₇-alkyl or benzyl, Ar is aryl¹ or heteroaryl¹, m, n are independently from each other 1, 2 or 3.

Acids II-2 or II-3, wherein n and m=1, can alternatively be prepared via the route described in scheme 3: Dipolar 2+3 cycloaddition of aryl-propynoic acid ester XI with an azomethine ylide formed in situ under the reaction conditions from X leads to IX-2, which can either be saponified directly in the usual manner to acid II-2 or transformed into II-3 after change of the protecting group and saponification.

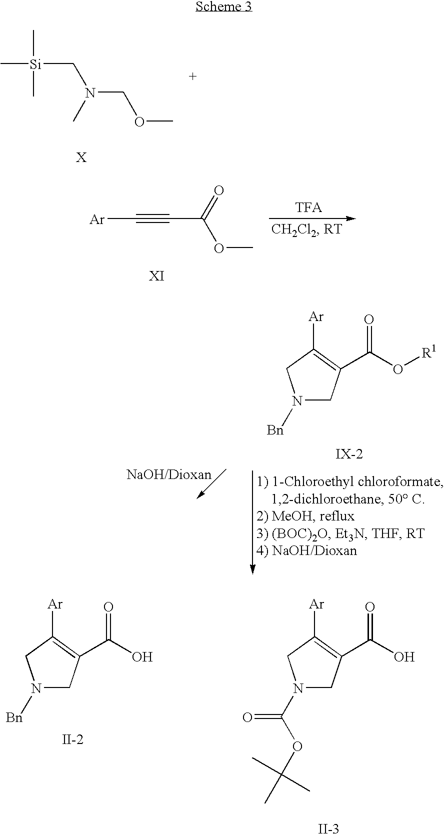

wherein R¹ is C₁₋₇-alkyl or benzyl and Ar is as defined above.

Acid II-4 is prepared via the route described in scheme 4: Dipolar 3+2 cycloaddition of thiole XIV with the phosphonic ester XVI to give thiophene XVII, which is saponified directly in the usual manner to acid II-4.

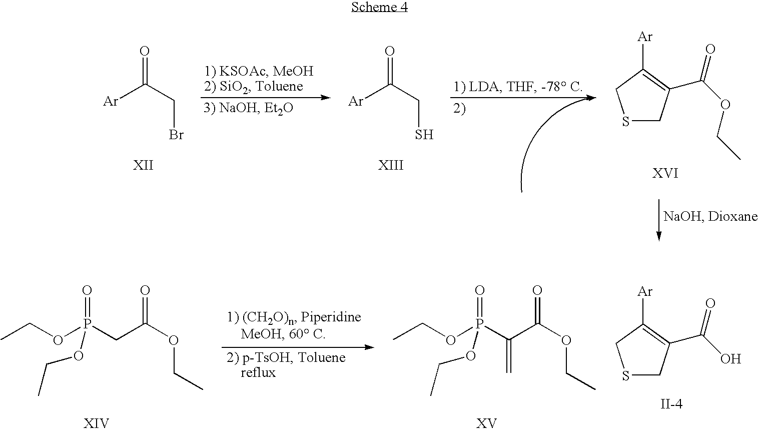

The invention may be described in detail as follow:

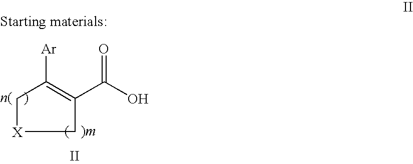

Tetrasubstituted acids II, e.g., can be the following:
2-aryl/heteroaryl-cyclopent-1-ene carboxylic acids,
4-aryl/heteroaryl-2,5-dihydro-1H-pyrrole-3-carboxylic acids,
4-aryl/heteroaryl-2,5-dihydro-furan-3-carboxylic acids,
4-aryl/heteroaryl-2,5-dihydro-thiophene-3-carboxylic acids,
1,1-dioxo-4-aryl-2,5-dihydro-1H-1λ⁶-thiophene-3-carboxylic acids,
2-aryl/heteroaryl-cyclohexyl-1-ene carboxylic acid,
4-aryl/heteroaryl-1,2,5,6-tetrahydro-pyridine-3-carboxylic acids,
5-aryl/heteroaryl-1,2,3,6-tetrahydro-pyridine-4-carboxylic acids,
4-aryl/heteroaryl-5,6-dihydro-2H-pyran-3-carboxylic acids,
5-aryl/heteroaryl-3,6-dihydro-2H-pyran-4-carboxylic acids,
4-aryl/heteroaryl-5,6-dihydro-2H-thiopyran-3-carboxylic acids,
5-aryl/heteroaryl-3,6-dihydro-2H-thiopyran-4-carboxylic acids,
1,1-dioxo-4-aryl/heteroaryl-1,2,5,6-tetrahydro-1λ⁶-thiopyran-3-carboxylic acids,
1,1-dioxo-5-aryl/heteroaryl-1,2,3,6-tetrahydro-1λ⁶-thiopyran-4-carboxylic acids,
1-oxo-4-aryl/heteroaryl-1,2,5,6-tetrahydro-1λ⁴-thiopyran-3-carboxylic acids,
1-oxo-4-aryl/heteroaryl-2,5-dihydro-1H-1λ⁴-thiophene-3-carboxylic acids,
2-phenyl-cyclohept-1-enecarboxylic acid or
2-phenyl-cyclooct-1-enecarboxylic acid and corresponding salts thereof.

### Products:

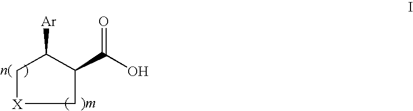

Acids I can be the following:
2-aryl/heteroaryl-cyclopentane carboxylic acids,
4-aryl/heteroaryl-2,5-dihydro-1H-pyrrolidine-3-carboxylic acids,
4-aryl/heteroaryl-tetrahydrofuran-3-carboxylic acids,
4-aryl/heteroaryl-tetrahydro-thiophene-3-carboxylic acids,
1,1-dioxo-4-aryl/heteroaryl-tetrahydro-1λ⁶-thiophene-3-carboxylic acids,
1-oxo-4-aryl/heteroaryl-tetrahydro-1λ⁴-thiophene-3-carboxylic acids,
2-aryl/heteroaryl-cyclohexane carboxylic acid,
4-aryl/heteroaryl-piperidine-3-carboxylic acids,
5-aryl/heteroaryl-piperidine-4-carboxylic acids,
4-aryl/heteroaryl-tetrahydro-pyran-3-carboxylic acids,
5-aryl/heteroaryl-tetrahydro-pyran-4-carboxylic acids,
4-aryl/heteroaryl-tetrahydro-thiopyran-3-carboxylic acids,
5-aryl/heteroaryl-tetrahydro-thiopyran-4-carboxylic acids,
1,1-dioxo-4-aryl/heteroaryl-hexahydro-1′-thiopyran-3-carboxylic acids,
1,1-dioxo-5-aryl/heteroaryl-hexahydro-1λ⁶-thiopyran-4-carboxylic acids,
1-oxo-4-aryl/heteroaryl-hexahydro-1λ⁴-thiopyran-3-carboxylic acids,
2-phenyl-cycloheptane carboxylic acid, or
2-phenyl-cyclooctane carboxylic acid and corresponding salts thereof.

### Catalysts:

### Ruthenium Complex Catalysts:

In the ruthenium complex catalysts ruthenium is characterized by the oxidation number II. Such ruthenium complexes can optionally comprise further ligands, either neutral or anionic. Examples of such neutral ligands are e.g. olefins, e.g. ethylene, propylene, cyclooctene, 1,3-hexadiene, norbornadiene, 1,5-cyclooctadiene, benzene, hexamethylbenzene, 1,3,5-trimethylbenzene, p-cymene, or also solvents such as e.g. tetrahydrofuran, dimethylformamide, acetonitrile, benzonitrile, acetone, toluene and methanol. Examples of such anionic ligands are CH₃COO⁻, CF₃COO⁻ or halides. If the ruthenium complex is charged, non-coordinating anions such as halides, BF₄⁻, ClO₄⁻, SbF₆⁻, PF₆⁻, B(phenyl)₄⁻, B(3,5-di-trifluoromethyl-phenyl)₄⁻, CF₃SO₃⁻, C₆H₅SO₃⁻ are present.

Suitable ruthenium complexes in question can be represented e.g. by the following formula

Ru(Z)₂D XVII

[Ru(Z)₂₋ₚ(D)(L¹)ₘ](B)ₚ XVIII

wherein Z represents halogen or the group A-COO, A represents lower alkyl, aryl², halogenated lower alkyl or halogenated aryl², D represents a chiral diphosphine ligand, B represents a non-coordinating anion as defined above and L¹ represents a neutral ligand as defined above, p represents the numbers 1 and 2, the ligands can be the same or different, m represents the number 1, 2 or 3.

These complexes can in principle be manufactured in a manner known per se, e.g. according to B. Heiser et al., Tetrahedron: Asymmetry 1991, 2, 51 or N. Feiken et al., Organometallics 1997, 16, 537 or J.-P. Genet, Acc. Chem. Res. 2003, 36, 908 or K. Mashima et al., J. Org. Chem. 1994, 53, 3064 and references cited therein.

Conveniently and preferably, ruthenium complexes are manufactured, for example, by reacting a complex of the formula

[Ru(Z¹)₂(L¹)ₘ]ₚ(H₂O)_{q} XIX

wherein Z¹ represents halogen or a group A¹-COO, A¹ represents lower alkyl or halogenated lower alkyl, L¹ represents a neutral ligand as defined above, m represents the number 1, 2 or 3, p represents the number 1 or 2 and q represents the number 0 or 1, with a chiral diphosphine ligand. Where m represents the number 2 or 3, the ligands can be the same or different.

Typically, ruthenium catalysts exemplified within the present invention can be prepared according to the method described by M. P. Fleming et al., U.S. Pat. No. 6,545,165 B1, for the preparation of chiral ruthenium dicarboxylate diphosphines.

### Rhodium Complex Catalysts:

In the rhodium complex catalysts rhodium is characterized by the oxidation number I, and contains a chiral phosphine ligand. Such rhodium complexes can optionally comprise further ligands, either neutral or anionic.

Examples of such neutral ligands are e.g. olefins, e.g. ethylene, propylene, cyclooctene, 1,3-hexadiene, 1,5-hexadiene, norbornadiene (nbd=bicyclo-[2.2.1]hepta-2,5-diene), (Z,Z)-1,5-cyclooctadiene (cod) or other dienes which form readily soluble complexes with rhodium or ruthenium, benzene, hexamethylbenzene, 1,3,5-trimethylbenzene, p-cymene, or also solvents such as e.g. tetrahydrofuran, dimethylformamide, acetonitrile, benzonitrile, acetone, methanol and pyridine.

Examples of such anionic ligands are halides or the group A-COO⁻, wherein A represents lower alkyl, aryl², halogenated lower alkyl or halogenated aryl². Preferably, A-COO is CH₃COO⁻ or CF₃COO⁻. If the rhodium complex is charged, non-coordinating anions such as a halide, BF₄⁻, ClO₄⁻, SbF₆⁻, PF₆⁻, B(phenyl)₄⁻, B(3,5-di-trifluoromethyl-phenyl)₄⁻, CF₃SO₃⁻, C₆H₅SO₃⁻ are present.

Preferred catalysts comprising rhodium and a chiral diphosphine are of the formula

[Rh(chiral diphosphine)LX] or [Rh(chiral diphosphine)L]⁺A⁻

wherein X is a halide such as Cl⁻, Br⁻ or I⁻, L is a neutral ligand as defined above and A is an anion of an oxyacid or a complex acid such as ClO₄, PF₆, BR₄; wherein R is halogen or aryl², SbF₆ or AsF₆. If L is a ligand comprising two double bonds, e.g. 1,5-cyclooctadiene, only one such L is present. If L is a ligand comprising only one double bond, e.g. ethylene, two such L are present.

A rhodium complex catalyst can be prepared, for example, by reaction of rhodium precursors such as e.g. di-η⁴-chloro-bis[η⁴-(Z,Z)-1,5-cyclo-octadiene]dirhodium(I) ([Rh(cod)Cl]₂), di-μ-chloro-bis[η⁴-norbornadiene]-dirhodium(I) ([Rh(nbd)Cl]₂), bis[η⁴-(Z,Z)-1,5-cyclooctadiene]rhodium tetra-fluoroborate ([Rh(cod)₂]BF₄) or bis [⁴-(Z,Z)-cyclooctadiene]rhodium perchlorate ([Rh(cod)₂]ClO₄) with a chiral diphosphine ligand in a suitable inert organic or aqueous solvent (e.g. according to the methods described in Experimental Chemistry, 4^{th} edition, Vol. 18, Organometallic complexes, pp. 339-344, Ed. Chemical Society of Japan, 1991, Maruzen or J. Am. Chem. Soc. 1971, 93, 2397 or E. Jacobsen, A. Pfaltz, H. Yamamoto (Eds.), Comprehensive Asymmetric Catalysis I-III, Springer Verlag Berlin (1999) and references cited therein).

Rhodium or ruthenium complex catalysts as described above can also be prepared in situ, i.e. just before use and without isolation. The solution in which such a catalyst is prepared can already contain the substrate for the enantioselective hydrogenation or the solution can be mixed with the substrate just before the hydrogenation reaction is initiated.

The chiral diphosphine ligand is characterized by formula (3), (4), (5), (6), (7), (8), (9), (10), (11), (12), (13), (14), (15) or (16).

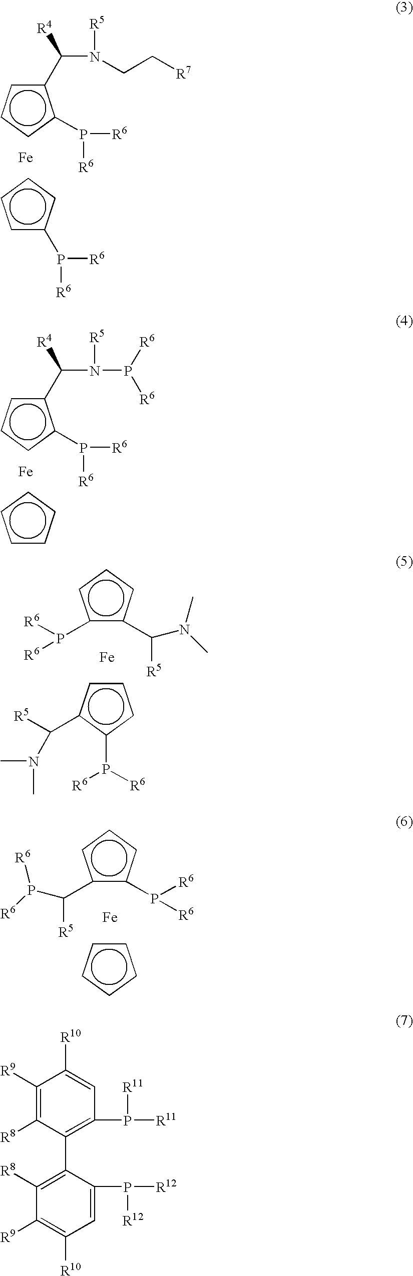

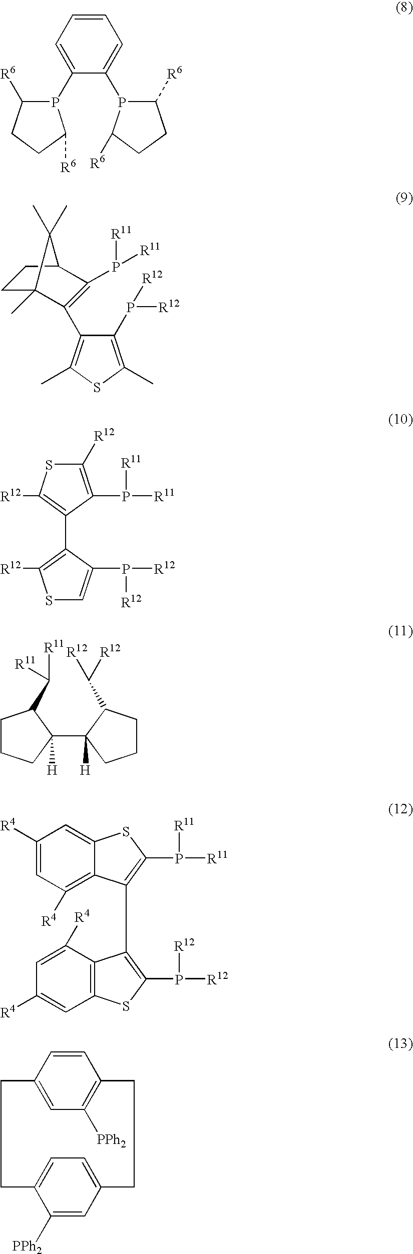

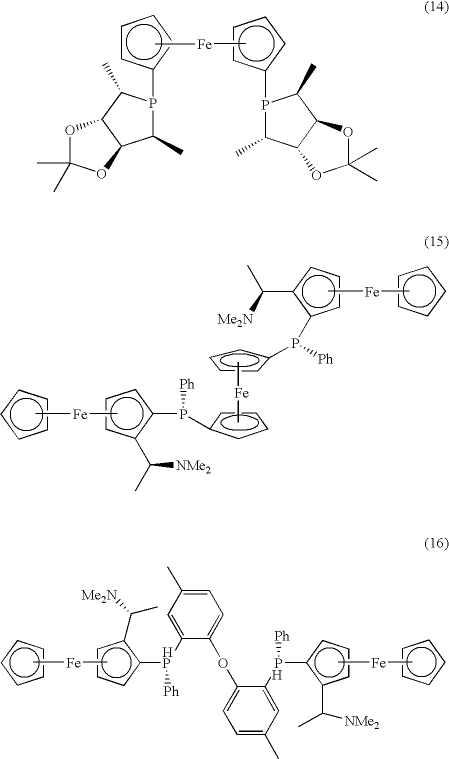

wherein

### R⁴ is lower-alkyl;

### R⁵ is lower-alkyl;

### R⁶ independently is aryl², heteroaryl², cycloalkyl or lower-alkyl;

### R⁷ is N(lower-alkyl)₂ or piperidinyl;

### R⁸ is lower-alkyl, lower-alkoxy, hydroxy or lower-alkyl-C(O)O—;

### R⁹ and R¹⁰ are each independently hydrogen, lower-alkyl, lower-alkoxy or di(lower-alkyl)amino; or

R⁸ and R⁹ which are attached to the same phenyl group, or R⁹ and R¹⁰ which are attached to the same phenyl group, or both R⁸, taken together, are —X—(CH₂)ᵣ—Y—, wherein X is —O— or —C(O)O—, Y is —O— or —N(lower-alkyl)- and r is an integer from 1 to 6, or a CF₂ group; or

### R⁸ and R⁹, or R⁹ and R¹⁰, together with the carbon atoms to which they are attached, form a naphthyl, tetrahydronaphthyl or dibenzofuran ring;

R¹¹ and R¹² are each independently lower alkyl, cycloalkyl, phenyl, napthyl or heteroaryl, substituted with 0 to 7 substituents independently selected from the group consisting of lower-alkyl, lower-alkoxy, di(lower-alkyl)amino, morpholino, phenyl and tri(lower-alkyl)silyl;

If R¹¹ is phenyl, it is substituted with 0 to 5, preferably 0 to 3 substituents as described above.

In a more preferred embodiment, the catalyst is of the formula Ru(Z)₂D, wherein the chiral diphosphine is characterized by formula (7), (9), (10) or (12) and wherein Z is CH₃COO, CF₃COO or a halogenide.

Preferably, the chiral diphosphine is selected from the group consisting of (R) and (S)-enantiomers: MeOBIPHEP, BIPHEMP, TMBTP, 2-Naphthyl)-MeOBIPHEP, (6-MeO-2-Naphthyl)-MeOBIPHEP, 2-(Thienyl)-MeOBIPHEP, 3,5-tBu-MeOBIPHEP, PHANEPHOS, BICP, TriMeOBIPHEP, (R,R,S,S)-Mandyphos, BnOBIPHEP, BenzoylBIPHEP, pTol-BIPHEMP, tButylCOOBIPHEP, iPrOBIPHEP, p-Phenyl-MeOBIPHEP, pAn-MeOBIPHEP, pTol-MeOBIPHEP, 3,5-Xyl-MeOBIPHEP, 3,5-Xyl-BIPHEMP, BINAP and 2-Furyl-MeOBIPHEP, 3,5-Xyl-4-MeO-MeOBIPHEP, 2-Furyl-MeOBIPHEP, BITIANP, DuanPHos, C2-Tunaphos, f-BINAPHANE, Stylacat 4/1, TOLFER Stylacat 4/2 or Stylacat 3/1/1. More preferably, the chiral diphosphine is ((S)-(6-MeO-2-Naphthyl)-MeOBIPHEP, 3,5-Xyl-4-MeO-MeOBIPHEP, (S)-2-Furyl-MeOBIPHEP or BITIANP. Each of these chiral diphosphines individually constitutes a preferred embodiment of the present invention.

### Solvents for Ruthenium Complexes:

Alcohols, hydrocarbons, chlorinated hydrocarbons, supercritical or liquid carbon dioxide, THF or water. Preferred solvents are alcohols.

### Solvents for Rhodium Complexes:

Alkanols or aromatic hydrocarbons, such as benzene, toluene, trifluoro toluene, or halogenated hydrocarbons, such as dichloromethane, dichlororethane, etc., or polyalcohols such as ethylene glycol, or amides such as DMF, DMA, N-methylpyrrolidinone, or supercritical or liquid carbon dioxide, acetonitrile or DMSO.

The solvents can be used alone or as mixture of solvents mentioned above. The concentration of solvents is 1-50 W %, preferentially 5-20%.

### Additives:

### Bases: tertiary amines, such as NEt₃, i-Pr₂NEt,

secondary amines, such as iPr₂NH,
primary amines, such as C₆H₅CH₂NH₂, 1-phenyl-benzylamine, (R) or (S)),
diamines, such as ethylene diamine, tetramethylethylene diamine, salts of carboxylic acids, such as NaOAc, salts of alcoholates, such as NaOEt, or salts of NaOH. tetrasubstituted ammonium salts, such as Bu₄NX (X═F, Cl, Br, I)

Preferred additives are tertiary amines as described above.

The amount of base is in the range of 0.1-100 equivalents, preferentially 0.1-1.2 molar equivalents. Most preferred range is 0.15-1 molar equivalent.

### Reaction Conditions:

### Pressure: 1-150 bar, preferentially 10-100 bar.

### Temperature: 10-100° C., preferentially 20-80° C.

### Substrate/catalyst ratio (s/c): 5-30000, preferentially 100-10000

### General Description

With regard to the invention, the process for the preparation of enantiomerically enriched cyclic β-arylcarboxylic acid derivatives of formula

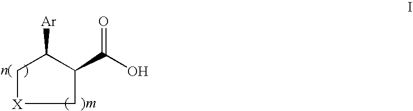

comprises catalytic homogeneous enantioselective hydrogenation of a compound of formula (II)

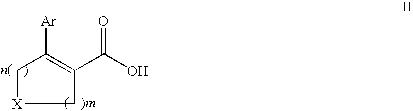

wherein
X is —C(R)(R′)—, —N(R″)—, —O—, —S(O)ₒ—, C(O)N(R″), —N(R″)C(O)— or —C(O)—;
R and R′ are each independently hydrogen, C₁₋₇-alkyl, C₁₋₇-alkyl substituted by halogen, C₁₋₇-alkoxy, hydroxy or —(CH₂)ₚ—Ar;
R″ is hydrogen, C₁₋₇-alkyl, C₁₋₇-alkyl substituted by halogen, —S(O)ₙ—C₁₋₇-alkyl, —S(O)ₒ—Ar, —S(O)ₙ—NRR′, —(CH₂)ₚ—Ar, —C(O)—C₁₋₇-alkyl, —C(O)—Ar, —C(O)—NRR′ or —C(O)O—C₁₋₇-alkyl;
Ar is aryl¹ or heteroaryl¹;
n is 0, 1, 2 or 3;
m is 0, 1, 2 or 3;
o is 0, 1 or 2;
p is 0, 1, or 2;
   and corresponding salts thereof
   in the presence of a catalyst comprising

Ru(Z)₂D XII

wherein Z represents halogen or the group A-COO, A represents lower alkyl, aryl², halogenated lower alkyl or halogenated aryl² and D represents a chiral diphosphine ligand, or comprises

[Rh(chiral diphosphine)LX] or [Rh(chiral diphosphine)L]⁺A⁻

wherein X is Cl⁻, Br⁻ or I⁻, L is a neutral ligand, selected from the group consisting of ethylene, propylene, cyclooctene, 1,3-hexadiene, norbornadiene, 1,5-cyclooctadiene, benzene, hexamethylbenzene, 1,3,5-trimethylbenzene, p-cymene, tetrahydrofuran, dimethylformamide, acetonitrile, benzonitrile, acetone or methanol,

### A is an anion of an oxyacid or a complex acid selected from the group consisting of ClO₄, PF₆, and BR₄, wherein R is halogen or aryl, SbF₆ or AsF₆,

to yield said compound of formula (I).

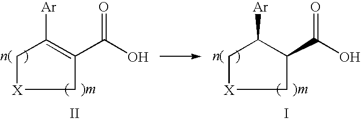

In a glove box an autoclave equipped with a glass insert and a magnetic stirring bar is charged with a compound of formula II, for example with 2-phenyl-cyclohex-1-ene-carboxylic acid, with a ruthenium catalyst, such as [Ru(OAc)₂((R)-2-furyl)-MeOBIPHEP], with an additive, for example triethylamine and a solvent, such as methanol. The asymmetric hydrogenation is run for about 42 h at 20-80° C. under 40 bar of hydrogen. After cooling to room temperature the pressure is released from the autoclave, the solvent is diluted with tert-butyl methyl ether, extracted, dried and concentrated in vacuo to give a compound of formula I, for example (−)-2-phenyl-cyclohexane carboxylic acid.

Enantiomeric excess (ee) values were determined by chiral GC or HPLC.

### Experimental:

List of abbreviations for the used ligands:
BIPHEMP¹ (6,6′-Dimethylbiphenyl-2,2′-diyl)bis(diphenylphosphine)
pTol-BIPHEMP¹ (6,6′-Dimethylbiphenyl-2,2′-diyl)bis(di-p-tolylphosphine)
3,5-Xyl-BIPHEMP¹ Phosphine, [6,6′-dimethoxy[1,1′-biphenyl]-2,2′-diyl]bis [bis(3,5-dimethylphenyl)-
MeOBIPHEP¹ (6,6′-Dimethoxybiphenyl-2,2′-diyl)bis(diphenylphosphine)
(2-Naphthyl)-(6,6′-Dimethoxybiphenyl-2,2′-diyl)bis(di-2-naphthylphosphin)
MeOBIPHEP¹
6-MeO-2-Naphthyl- (6,6′-Dimethoxybiphenyl-2,2′-diyl)bis(di-2-(6-methoxy)-
MeOBIPHEP¹ naphthylphosphine)
3,5-Xyl,4-MeO- (6,6′-Dimethoxy[1,1′-biphenyl]-2,2′-diyl)bis [bis(3,5-di-tert-
MeOBIPHEP¹ butyl-4-methoxyphenyl)phosphine)
3,5-t-Bu-MeOBIPHEP¹ (6,6′-Dimethoxy[1,1′-biphenyl]-2,2′-diyl)bis [bis(3,5-di-tert-butyl-phenyl)phosphine)
2-Furyl-MeOBIPHEP¹ (6,6′-Dimethoxybiphenyl-2,2′-diyl)bis(di-2-furylphosphine)
2-Thienyl- (6,6′-Dimethoxy[1,1′-biphenyl]-2,2′-diyl)bis(bis(2-
MeOBIPHEP¹ thienyl)phosphine)
pPhenyl-MeOBIPHEP¹ (6,6′-dimethoxy[1,1′-biphenyl]-2,2′-diyl)bis [bis([1,1′-biphenyl]-4-yl)-phosphine
pAn-MeOBIPHEP¹ (6,6′-dimethoxy[1,1′-biphenyl]-2,2′-diyl)bis [bis(4-methoxyphenyl)-phosphine
pTol-MeOBIPHEP¹ (6,6′-Dimethoxybiphenyl-2,2′-diyl)bis [di(p-tolyl)phosphine]
3,5-Xyl-MeOBIPHEP¹ [6,6′-Dimethoxy[1,1′-biphenyl]-2,2′-diyl]bis [bis(3,5-dimethylphenyl)phosphine
   (CAS Reg. No. 394248-45-4 (R))
TriMeOBIPHEP¹ Phosphine, (4,4′,5,5′,6,6′-hexamethoxy[1,1′-biphenyl]-2,2′-diyl)bis [diphenyl]
BenzoylBIPHEP⁶ (6,6′-Dibenzoyloxybiphenyl-2,2′-diyl)bis(diphenylphosphin)
tButylCOOBIPHEP¹ Propanoic acid, 2,2-dimethyl-,6,6′-bis(diphenylphosphino) [1,1′-biphenyl]-2,2′-diyl ester
iPrOBIPHEP¹ (6,6′-Di-2-propoxybiphenyl-2,2′-diyl)bis(diphenylphosphin)
BnOBIPHEP¹ (6,6′-Dibenzyloxybiphenyl-2,2′-diyl)bis(diphenylphosphin)
BINAP 2,2′-Bis(diphenylphosphino)-1,1′-binaphthyl
   (commercially available from Fluka)
DIOP 1,4-Bis-(diphenylphosphino)-1,4-dideoxy-2,3-O.isopropylidene-threitol (commercially available from Fluka)
BITIANP² 3,3′-bis-diphenylphosphanyl-1H, 1′H-[4,4′]-biisothiochromenyl
BICP³ 2,2′-bis(diphenylphosphino)-(1S,1′S,2S,2′S)-1,1′-bicyclopentyl
DuanPhos³ 2,2′-Di-tert-butyl-2,3,2′,3′-tetrahydro-1H, 1′H-(1,1′)-biisophosphinolyl
C2-Tunaphos³ (6,6′-O-[1,2-ethylene]-oxybiphenyl-2,2′-diyl)-bis(diphenyl)phosphine
f-BINAPHANE³ 1,1′-Bis-((S)-4,5-dihydro-3H-dinaphtho[2,1-c: 1′,2′-e]phosphepino)-ferrocene
TMBTP⁵ 2,2′,5,5′-Tetramethyl-4,4′-bis(diphenylphosphino)-3,3′-bithiophene
Mandyphos⁴ 1,1′-Bis [(dimethylamino)phenylmethyl]-2,2′-bis(diphenyl-diphosphino)-ferrocene
Stylacat 4/1⁷ 1,1′-bis-[((1-N,N-Dimethylamino)ethylferrocenyl)-(phenylphosphino)]ferrocene
TOLFER 2,2′-(bis-[((1-N,N-Dimethylamino)ethylferrocenyl)-
Stylacat 4/2⁷ phenylphosphino]-4-tolylether
Stylacat 3/1/1⁷ 2-[1-[(N-Methyl-N-diphenylphosphino)amino]ethyl]-1-[(1-naphthyl)phenylphosphino]ferrocene
¹⁾These ligands are known and/or can be prepared according to the examples or methods as described in patent application documents EP 0 398 132, WO 92/16535, EP 0 104 375 or EP 0 580 331.
²Synthesis and characterization described in: Benincori, T.; Brenna, E.; Sannicolo, F.; Trimarco, L.; Antognazza, P.; Cesarotti, E.; Demartin, F.; Pilati, T. J. Org. Chem. 1996, 61, 6244.
³Commercially available from Chiral Quest Inc., Princeton Corporate Plaza, Monmouth Jct., N.J. 08852, USA
⁴Commercially available from Strem Chemicals Inc. D-77672 Kehl
⁵Commercially available from Chemi S.p. A., Via dei Lavoratori, Cinasello Balsamo, Milano 20092, Italy.
⁶Synthesis according to: Bulliard, Michel; Laboue, Blandine; Roussiasse, Sonia. Use of optically active acyloxy-substituted diphosphinobiphenyls as ligands for catalyzed asym. hydrogenation or isomerization, WO 2002012253 A1.
⁷Commercially available from Phoenix Chemicals, 34 Thursby Road, Croft Business Park, Bromborough, Wirral, Merseyside CH62 3PW, UK.

### Enantioselective Hydrogenations

### EXAMPLE 1 OF I

### (+)-(3R,4R)-4-(4-Fluoro-phenyl)-piperidine-1,3-dicarboxylic acid-1-tert-butyl ester and (−)-(3S,4S)-4-(4-Fluoro-phenyl)-piperidine-1,3-dicarboxylic acid 1-tert-butyl ester

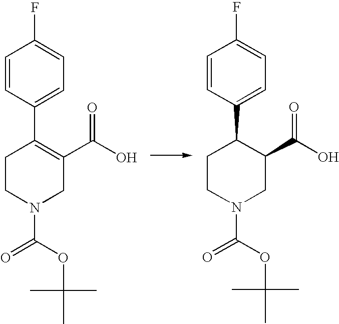

In a glove box (O₂ content≦2 ppm) a 35 ml autoclave equipped with a 15 ml glass insert and a magnetic stirring bar was charged with 0.300 g (0.934 mmol) of 4-(4-fluoro-phenyl)-5,6-dihydro-2H-pyridine-1,3-dicarboxylic acid-1-tert-butyl ester, 9.67 mg (0.00936 mmol) of [Ru(OAc)₂((S)-3,5-Xyl-4-MeO)-MeOBIPHEP], 15 mg (0.16 mmol, 0.16 eq.) of triethylamine and 5 ml of methanol. The asymmetric hydrogenation was run for 42 h at 80° C. under 40 bar of hydrogen. After cooling to room temperature the pressure was released from the autodave, the methanol solution was diluted with 50 ml of tert-butyl methyl ether and extracted with two 50-ml portions of a 1 M aqueous sodium hydroxide solution. The aqueous layer was poured on ice, acidified with ice-cold 2 M aqueous hydrochloric acid solution to pH 1 and extracted with two 100-ml portions of ethyl acetate. The combined organic layers were dried over sodium sulfate, filtered and concentrated in vacuo to give (+)-(3R,4R)-4-(4-fluoro-phenyl)-piperidine-1,3-dicarboxylic acid-1-tert-butyl ester in 89% yield (0.27 g) and with 96.6% ee.

MS m/e (%): 322 (M−H⁺, 100).

### GC Method for ee Determination:

A 2-mg sample of the title compound was converted to the methyl ester by treatment with 0.5 ml of an approximately 0.5 M solution of diazomethane in diethyl ether at room temperature. After evaporation of excess diazomethane and diethyl ether under a gentle stream of argon the residue was dissolved in 1 ml of ethyl acetate. BGB-175 column, 10 m*0.1 mm*df 0.1 μm, hydrogen 230 kPa, split ratio 1:300; temperature gradient 100-200° C., program with 2° C./min; injector temperature 200° C., detector temperature 210° C. Retention times: 46.59 min (methyl ester of (+)-acid), 46.76 min (methyl ester of (−)-acid).

The absolute configuration was assigned as described below after transformation of the title compound to its trans isomer (−)-(3S,4R)-4-(4-fluoro-phenyl)-piperidine-1,3-dicarboxylic acid 1-tert-butyl ester (reaction sequence described in examples 1 of III and I of V).

In a similar manner, but in a 6 ml, 35 ml or 185 ml autoclave, the reactions in Table 1 were performed.

TABLE 1

| Reaction | Scale | | | NEt₃ | t | Yield | Major | e.e. |
|---|---|---|---|---|---|---|---|---|
| No. | (g) | S/C | Catalyst | (equiv.) | (h) | (%) | enantiomer | (%) |
| 1^{a)} | 0.05 | 25 | Ru(OAc)₂((R)- | 0.6 | 42 | 90 | (−)^{d)} | 94.6 |
| | | | MeOBIPHEP) + 0.86 | | | | | |
| | | | toluene | | | | | |
| 2^{a)} | 0.05 | 25 | Ru(OAc)₂((S)-(6- | 0.6 | 42 | 80 | (+)^{e)} | 95.8 |
| | | | MeO-2-Naphtyl)- | | | | | |
| | | | MeOBIPHEP) | | | | | |
| 3^{a)} | 0.05 | 25 | Ru(OAc)₂((R)-3,5- | 0.6 | 42 | 88 | (−) | 93.8 |
| | | | tBu-MeOBIPHEP) | | | | | |
| 4^{a)} | 0.05 | 25 | Ru(OAc)₂((+)-(S)- | 0.6 | 42 | 84 | (+) | 88.5 |
| | | | TMBTP | | | | | |
| 5^{a)} | 0.05 | 25 | Ru(OAc)₂((S)-3,5- | 0.6 | 42 | 88 | (+) | 94.5 |
| | | | Xyl,4-MeO- | | | | | |
| | | | MeOBIPHEP) | | | | | |
| 6^{a)} | 0.05 | 25 | Ru (OAc)₂((all-S)- | 0.6 | 42 | 84 | (+) | 82.3 |
| | | | BICP) | | | | | |
| 7^{b)} | 0.3 | 100 | Ru(OAc)₂((S)-(6- | 0.16 | 42 | 90 | (+) | 91.5 |
| | | | MeO-2-Naphtyl)- | | | | | |
| | | | MeOBIPHEP) | | | | | |
| 9^{b)} | 0.3 | 100 | Ru(OAc)₂((S)- | 0.16 | 42 | 90 | (+) | 92.8 |
| | | | MeOBIPHEP) + 1.072 | | | | | |
| | | | toluene | | | | | |
| 11^{b)} | 0.3 | 250 | Ru(OAc)₂((S)-(6- | 0.16 | 42 | 87 | (+) | 94.7 |
| | | | MeO-2-Naphtyl)- | | | | | |
| | | | MeOBIPHEP) | | | | | |
| 12^{b)} | 0.3 | 250 | Ru(OAc)2((S)-3,5- | 0.06 | 42 | 83 | (+) | 95.7 |
| | | | Xyl,4-MeO- | | | | | |
| | | | MeOBIPHEP) | | | | | |
| 13^{c)} | 9.18 | 250 | Ru(OAc)₂((S)-3,5- | 0.06 | 42 | 94 | (+) | 94.6 |
| | | | Xyl,4-MeO- | | | | | |
| | | | MeOBIPHEP) | | | | | |
| 15^{c)} | 2.2 | 250 | Ru(OAc)₂((S)-3,5- | 1 | 42 | 99 | (+) | 95.3 |
| | | | Xyl,4-MeO- | | | | | |
| | | | MeOBIPHEP) | | | | | |

^{a)}35 ml autoclave.
^{b)}6 ml autoclave.
^{c)}185 ml autoclave.
^{d)}[α]_{D} = −54.44 (c = 0.369, CHCl₃).
^{e)}[α]_{D} = +56.26 (c = 0.446, CHCl₃).

### EXAMPLE 2 OF I

### (−)-4-(1H-Indol-3-yl)-piperidine-1,3-dicarboxylic acid-1-tert-butyl ester and (+)-4-(1H-Indol-3-yl)-piperidine-1,3-dicarboxylic acid-1-tert-butyl ester

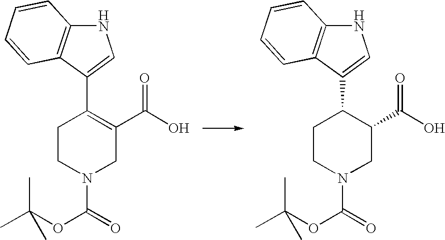

In a glove box (O₂ content≦2 ppm) a 185 ml autoclave equipped with a mechanical stirrer was charged with 1.00 g (2.92 mmol) of 4-(1H-indol-3-yl)-5,6-dihydro-2H-pyridine-1,3-dicarboxylic acid-1-tert-butyl ester, 8.88 mg (0.0117 mmol) of [Ru(OAc)₂((R)-2-furyl)-MeOBIPHEP], 295 mg (2.92 mmol, 1.0 eq.) of triethylamine and 20 ml of methanol. The asymmetric hydrogenation was run for 42 h at 80° C. under 40 bar of hydrogen. After cooling to room temperature the pressure was released from the autoclave, the methanol solution was diluted with 200 ml of tert-butyl methyl ether and extracted with two 100 ml portions of a 1 M aqueous sodium hydroxide solution. The aqueous layer was poured on ice, acidified with ice-cold 2 M aqueous hydrochloric acid solution to pH 1 and extracted with three 200-ml portions of ethyl acetate. The combined organic layers were dried over sodium sulfate, filtered and concentrated in vacuo to give (−)-4-(1H-indol-3-yl)-piperidine-1,3-dicarboxylic acid-1-tert-butyl ester in 89% yield and with 98.8% ee.

MS m/e (%): 245 (M+H⁺, 19).

### HPLC Method for ee Determination:

Chiralpak-OD-H column, 25 cm*4.6 mm, 90% n-heptane and 10% ethanol with 1% trifluroacetic acid, flow 0.8 ml/min, 25° C., 0.002 ml injection volume, 222 nm. Retention times: (−)-acid 13.4 min, (+)-acid 21.6 min.

In a similar manner, but in a 6 ml or 185 ml autoclave, the reactions in Table 2 were performed.

TABLE 2

| Reaction | Scale | | | NEt₃ | | Yield | Major | e.e. |
|---|---|---|---|---|---|---|---|---|
| No. | (g) | S/C | Catalyst | (equiv.) | t (h) | (%) | enantiomer | (%) |
| 1^{a)} | 0.05 | 25 | Ru(OAc)₂((rac)- | 1 | 42 | 60 | racemate | — |
| | | | BIPHEMP) | | | | | |
| 2^{a)} | 0.05 | 25 | Ru(OAc)₂((R)-(2- | 1 | 42 | 80 | (−)^{c)} | 99.0 |
| | | | Furyl)- | | | | | |
| | | | MeOBIPHEP) | | | | | |
| 3^{a)} | 0.05 | 25 | Ru(OAc)₂((S)-3,5- | 1 | 42 | 80 | (+)^{d)} | 95.0 |
| | | | Xyl,4-MeO- | | | | | |
| | | | MeOBIPHEP) | | | | | |
| 4^{a)} | 0.05 | 25 | Ru(OAc)₂((S)-(6- | 1 | 42 | 80 | (+) | 95.6 |
| | | | MeO-2-Naphtyl)- | | | | | |
| | | | MeOBIPHEP) | | | | | |
| 6^{b)} | 1.00 | 250 | Ru(OAc)₂((S)-(6- | 1 | 42 | 92 | (+) | 94.0 |
| | | | MeO-2-Naphtyl)- | | | | | |
| | | | MeOBIPHEP) | | | | | |

^{a)}6 ml autoclave.
^{b)}185 ml autoclave.
^{c)}[α]_{D} = −94.46 (c = 0.29, MeOH).
^{d)}[α]_{D} = +93.53 (c = 0.265 MeOH).

### EXAMPLE 3 OF I

### (−)-4-o-Tolyl-piperidine-1,3-dicarboxylic acid 1-tert-butyl ester and (+)-4-o-Tolyl-piperidine-1,3-dicarboxylic acid 1-tert-butyl ester

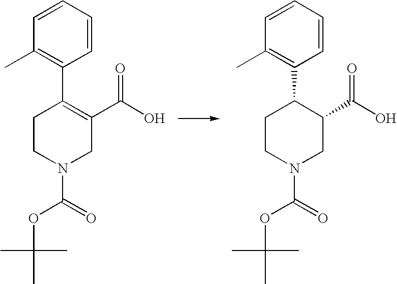

In a glove box (O₂ content≦2 ppm) a 35 ml autodave equipped with a 15 ml glass insert and a magnetic stirring bar was charged with 300 mg (0.945 mmol) of 4-o-tolyl-5,6-dihydro-2H-pyridine-1,3-dicarboxylic acid 1-tert-butyl ester, 7.2 mg (0.0094 mmol) of [Ru(OAc)₂((R)-(2-furyl)-MeOBIPHEP], 95.9 mg (0.945 mmol, 1.0 eq.) of triethylamine and 6 ml of methanol. The asymmetric hydrogenation was run for 42 h at 80° C. under 40 bar of hydrogen. After cooling to room temperature the pressure was released from the autoclave, the methanol solution was diluted with 100 ml of tert-butyl methyl ether and extracted with two 100-ml portions of a 1 M aqueous sodium hydroxide solution. The aqueous layer was poured on ice, acidified with ice-cold 2 M aqueous hydrochloric acid solution to pH 1 and extracted with three 100-ml portions of ethyl acetate. The combined organic layers were dried over sodium sulfate, filtered and concentrated in vacuo to give (−)-4-o-tolyl-piperidine-1,3-dicarboxylic acid 1-tert-butyl ester in 75% yield and with 99.1% ee. Crystallization from ethyl acetate/n-heptane gave (−)-4-o-tolyl-piperidine-1,3-dicarboxylic acid 1-tert-butyl ester with >99.9% ee.

MS m/e (%): 318 (M−H⁺, 100).

[α]_{D}=−79.03 (c=0.612, CHCl₃)

### HPLC Method for ee Determination:

Chiralpak-ADH column, 25 cm*4.6 mm, 85% n-heptane+15% ethanol with 1% trifluroacetic acid, flow 0.7 ml/min, 20° C., 0.005 ml injection volume, 215 nm. Retention times: (−)-acid 8.1 min, (+)-acid 8.8 min.

In a similar manner, but in a 6 ml or 35 ml autoclave, the reactions in Table 3 were performed.

TABLE 3

| Reaction | Scale | | | NEt₃ | | Yield | Major | e.e. |
|---|---|---|---|---|---|---|---|---|
| No. | (g) | S/C | Catalyst | (equiv.) | t (h) | (%) | enantiomer | (%) |
| 1^{a)} | 0.1 | 25 | Ru(OAc)₂((rac)- | 0.5 | 48 | 98 | racemate | — |
| | | | BIPHEMP) | | | | | |
| 2^{b)} | 0.05 | 25 | Ru(OAc)₂((R)- | 0.7 | 42 | 80 | (−) | 80.5 |
| | | | MeOBIPHEP) + 0.86 | | | | | |
| | | | toluene | | | | | |
| 3^{b)} | 0.05 | 25 | Ru(OAc)₂((S)-(6- | 0.7 | 42 | 80 | (+) | 82.9 |
| | | | MeO-2-Naphtyl)- | | | | | |
| | | | MeOBIPHEP) | | | | | |
| 4^{b)} | 0.05 | 25 | Ru(OAc)₂((R)-3,5- | 0.7 | 42 | 80 | (−) | 50 |
| | | | tBu-MeOBIPHEP) | | | | | |
| 5^{b)} | 0.05 | 25 | Ru(OAc)2((S)-3,5- | 0.7 | 42 | 80 | (+) | 76.2 |
| | | | Xyl,4-MeO- | | | | | |
| | | | MeOBIPHEP) | | | | | |
| 6^{b)} | 0.05 | 25 | Ru(OAc)₂((R)- | 1 | 66.5 | 80 | (−) | 90.8 |
| | | | MeOBIPHEP) + 0.86 | | | | | |
| | | | toluene | | | | | |
| 7^{b)} | 0.05 | 25 | Ru(OAc)₂((R)-(2- | 1 | 66.5 | 80 | (−) | 95.3 |
| | | | Furyl)- | | | | | |
| | | | MeOBIPHEP) | | | | | |
| 8^{b)} | 0.05 | 25 | Ru(OAc)2((R)- | 1 | 66.5 | 80 | (−) | 84.1 |
| | | | [2,2]- | | | | | |
| | | | PHANEPHOS | | | | | |
| 9^{b)} | 0.05 | 25 | Ru(OAc)₂((R)- | 1 | 66.5 | 80 | (−) | 93.4 |
| | | | BITIANP) | | | | | |
| 10^{b)} | 0.05 | 25 | Ru(OAc)₂((+)-(S)- | 1 | 66.5 | 80 | (+) | 51.1 |
| | | | TMBTP | | | | | |
| 11^{b)} | 0.05 | 25 | Ru(OAc)₂((S)-(2- | 1 | 66.5 | 80 | (+) | 82.3 |
| | | | Thienyl)- | | | | | |
| | | | MeOBIPHEP) | | | | | |
| 15^{a)} | 0.3 | 100 | Ru(OAc)₂((S)- | 1 | 68 | 98 | (+) | 95.5 |
| | | | BITIANP) | | | | | |

^{a)}35 ml autoclave.
^{b)}6 ml autoclave.

### EXAMPLE 4 OF I

### (+)-4-(3-Methoxy-phenyl)-piperidine-1,3-dicarboxylic acid-1-tert-butyl ester

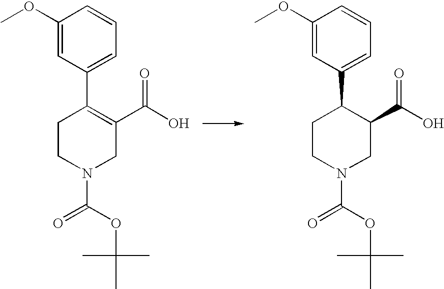

In a glove box (O₂ content≦2 ppm) a 6 ml autoclave equipped with a glass insert and a magnetic stirring bar was charged with 50 mg (0.15 mmol) of 4-(3-methoxy-phenyl)-5,6-dihydro-2H-pyridine-1,3-dicarboxylic acid-1-tert-butyl ester, 7.7 mg (0.0069 mmol) of [Ru(OAc)₂((S)-6-MeO-2-naphthyl)-MeOBIPHEP], 17.2 mg (0.172 mmol, 1.15 eq.) of triethylamine and 1 ml of methanol to give an orange suspension. The asymmetric hydrogenation was run for 66 h at 80° C. under 40 bar of hydrogen. After cooling to room temperature the pressure was released from the autoclave, the methanol solution was diluted with 30 ml of tert-butyl methyl ether and extracted with two 30-ml portions of a 1 M aqueous sodium hydroxide solution. The aqueous layer was poured on ice, acidified with ice-cold 2 M aqueous hydrochloric acid solution to pH 1 and extracted with two 50-ml portions of ethyl acetate. The combined organic layers were dried over sodium sulfate, filtered and concentrated in vacuo to give (+)-4-(3-methoxy-phenyl)-piperidine-1,3-dicarboxylic acid-1-tert-butyl ester in 80% yield and with 96.6% ee.

MS m/e (%): 334 (M−H⁺, 100).

[α]_{D}=+54.27 (c=0.387, CHCl₃)

### HPLC Method for ee Determination:

Chiralcel-OD-H column, 25 cm*4.6 mm, 90% n-heptane+10% ethanol with 1% trifluroacetic acid, flow 1 ml/min, 30° C., 0.002 ml injection volume, 215 nm, 266 nm. Retention times: (−)-acid 8.0 min, (+)-acid 11.0 min.

### EXAMPLE 5 OF I

### (+)-3-Phenyl-piperidine-1,4-dicarboxylic acid 1-tert-butyl ester and (−)-3-Phenyl-piperidine-1,4-dicarboxylic acid 1-tert-butyl ester

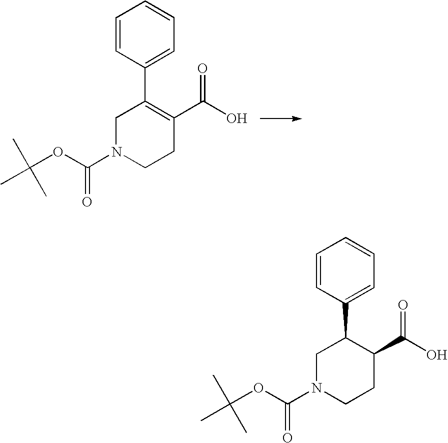

In a glove box (O₂ content≦2 ppm) a 35 ml autoclave equipped with a 15 ml glass insert and a magnetic stirring bar was charged with 400 mg (1.32 mmol) of 5-phenyl-3,6-dihxdro-2H-pyridine-1,4-dicarboxylic acid 1-tert-butyl ester, 14.7 mg (0.0131 mmol) of [Ru(OAc)₂((S)-6-MeO-2-naphthyl)-MeOBIPHEP], 133.1 mg (1.319 mmol, 1.0 eq.) of triethylamine and 8 ml of methanol. The asymmetric hydrogenation was run for 66 h at 80° C. under 40 bar of hydrogen. After cooling to room temperature the pressure was released from the autoclave, the methanol solution was diluted with 100 ml of tert-butyl methyl ether and extracted with two 100-ml portions of a 1 M aqueous sodium hydroxide solution. The aqueous layer was poured on ice, acidified with ice-cold 2 M aqueous hydrochloric acid solution to pH 1 and extracted with three 150-ml portions of ethyl acetate. The combined organic layers were dried over sodium sulfate, filtered and concentrated in vacuo to give (+)-3-phenyl-piperidine-1,4-dicarboxylic acid 1-tert-butyl ester in 100% yield and with 98.0% ee.

MS m/e (%): 304 (M−H⁺, 100).

[α]_{D}=+67.17 (c=0.636, CHCl₃)

### HPLC Method for ee Determination:

Chiralpak-IA column, 25 cm*4.6 mm, 50% n-heptane+50% (90% n-heptane+10% ethanol+0.1% trifluroacetic acid), flow 0.8 ml/min, 25° C., 0.002 ml injection volume, 215 nm. Retention times: (+)-acid 11.8 min, (−)-acid 12.8 min.

In a similar manner, but in a 6 ml or 35 ml autoclave, the reactions in Table 5 were performed.

TABLE 5

| Reaction | Scale | | | NEt₃ | | Yield | Major | |
|---|---|---|---|---|---|---|---|---|
| No. | (g) | S/C | Catalyst | (equiv.) | t (h) | (%) | enantiomer | e.e. (%) |
| 1^{a)} | 0.05 | 25 | Ru(OAc)₂((rac)- | 1 | 67 | 99 | racemate | — |
| | | | BIPHEMP) | | | | | |
| 2^{a)} | 0.05 | 25 | Ru(OAc)₂((S)3,5- | 1 | 48 | 100 | (+) | 98.8 |
| | | | Xyl,4-MeO- | | | | | |
| | | | MeOBIPHEP) | | | | | |
| 3^{a)} | 0.05 | 25 | Ru(OAc)₂((S)-(6- | 1 | 48 | 100 | (+) | 99.1 |
| | | | MeO-2-Naphtyl)- | | | | | |
| | | | MeOBIPHEP) | | | | | |
| 4^{a)} | 0.05 | 25 | Ru(OAc)₂((S)- | 1 | 48 | 100 | (+) | 98.0 |
| | | | BITIANP) | | | | | |
| 5^{b)} | 0.4 | 100 | Ru(OAc)₂((R)- | 1 | 66 | 100 | (−)^{c)} | 97.2 |
| | | | BITIANP) | | | | | |

^{a)}6 ml autoclave.
^{b)}35 ml autoclave.
^{c)}[α]_{D} = −65.19 (c = 0.515, CHCl₃).

### EXAMPLE 6 OF I

### (+)-4-Phenyl-piperidine-1,3-dicarboxylic acid 1-tert butyl ester and (−)-4-Phenyl-piperidine-1,3-dicarboxylic acid 1-tert butyl ester

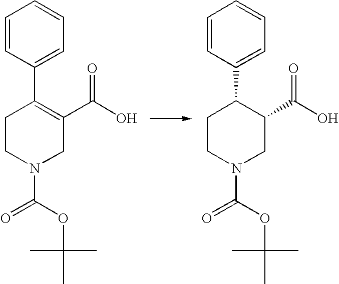

In a glove box (O₂ content≦2 ppm) a 35 ml autoclave equipped with a 15 ml glass insert and a magnetic stirring bar was charged with 0.300 g (0.989 mmol) of 4-phenyl-5,6-dihydro-2H-pyridine-1,3-dicarboxylic acid-1-tert-butyl ester, 3.01 mg (0.00396 mmol) of [Ru(OAc)₂((R)-2-Furyl)-MeOBIPHEP], 99 mg (0.989 mmol, 1 eq.) of triethylamine and 6 ml of methanol. The asymmetric hydrogenation was run for 68 h at 80° C. under 40 bar of hydrogen. After cooling to room temperature the pressure was released from the autoclave, the methanol solution was diluted with 50 ml of tert-butyl methyl ether and extracted with two 50-ml portions of a 1 M aqueous sodium hydroxide solution. The aqueous layer was poured on ice, acidified with ice-cold 2 M aqueous hydrochloric acid solution to pH 1 and extracted with two 100-ml portions of ethyl acetate. The combined organic layers were dried over sodium sulfate, filtered and concentrated in vacuo to give (−)-(4-phenyl)-piperidine-1,3-dicarboxylic acid-1-tert-butyl ester in 93% yield (0.28 g) and with 97.3% ee.

MS m/e (%): 306 (M+H⁺, 100%).

[α]_{D}=−59.80 (c=0.351, CHCl₃)

### GC Method for ee Determination:

A 2-mg sample of the title compound was converted to the methyl ester by treatment with 0.5 ml of an approximately 0.5 M solution of diazomethane in diethyl ether at room temperature. After evaporation of excess diazomethane and diethyl ether under a gentle stream of argon the residue was dissolved in 1 ml of ethyl acetate. BGB-172 column, 30 m*0.25 mm*df 0.25 μm, hydrogen 150 kPa, split ratio 1:20; temperature gradient 180-230° C., program with 2° C./min; injector temperature 210° C., detector temperature 240° C. Retention times: 19.90 min (methyl ester of (+)-acid), 20.23 min (methyl ester of (−)-acid).

In a similar manner, but with different chiral complexes, bases or solvents, the reactions in Table 6 were performed (all in 35 ml autoclaves).

TABLE 6

| Reaction | Scale | | | | Base | | Yield | Major | |
|---|---|---|---|---|---|---|---|---|---|
| No. | (g) | S/C | Catalyst | Solvent | 1 equiv. | t (h) | (%) | enantiomer | e.e. (%) |
| 1 | 0.1 | 25 | Ru(OAc)₂((S)- | MeOH | NEt₃ | 67 | 89 | (+) | 95.9 |
| | | | (6-MeO-2- | | | | | | |
| | | | Naphtyl)- | | | | | | |
| | | | MeOBIPHEP) | | | | | | |
| 2 | 0.1 | 25 | Ru(OAc)₂((S)- | MeOH | NEt₃ | 68 | 75 | (+) | 95.9 |
| | | | 3,5-Xyl,4-MeO- | | | | | | |
| | | | MeOBIPHEP) | | | | | | |
| 3 | 0.1 | 25 | Ru(OAc)₂((S)- | MeOH | NEt₃ | 68 | 88 | (+) | 96.5 |
| | | | (BITIANP) | | | | | | |
| 4 | 0.2 | 250 | [Ru(OAc)₂((R)- | MeOH | NEt₃ | 24 | 78 | (−) | 96.7 |
| | | | 2-Furyl)- | | | | | | |
| | | | MeOBIPHEP] | | | | | | |
| 5 | 0.05 | 25 | Ru(OAc)₂((S)- | MeOH | None | 68 | 40 | (+) | 94.3 |
| | | | (6-MeO-2- | | | | | | |
| | | | Naphtyl)- | | | | | | |
| | | | MeOBIPHEP) | | | | | | |
| 6 | 0.05 | 25 | Ru(OAc)₂((S)- | MeOH | Cs₂CO₃ | 68 | 98 | (+) | 96.6 |
| | | | (6-MeO-2- | | | | | | |
| | | | Naphtyl)- | | | | | | |
| | | | MeOBIPHEP) | | | | | | |
| 7 | 0.05 | 25 | Ru(OAc)₂((S)- | MeOH | NHEt₂ | 68 | 91 | (+) | 96.1 |
| | | | (6-MeO-2- | | | | | | |
| | | | Naphtyl)- | | | | | | |
| | | | MeOBIPHEP) | | | | | | |
| 8 | 0.05 | 25 | Ru(OAc)₂((S)- | MeOH | NaOEt | 68 | 80 | (+) | 96.2 |
| | | | (6-MeO-2- | | | | | | |
| | | | Naphtyl)- | | | | | | |
| | | | MeOBIPHEP) | | | | | | |
| 9 | 0.05 | 25 | Ru(OAc)₂((S)- | MeOH | NaCH(═O)H | 46 | 86 | (+) | 96.1 |
| | | | (6-MeO-2- | | | | | | |
| | | | Naphtyl)- | | | | | | |
| | | | MeOBIPHEP) | | | | | | |
| 10 | 0.05 | 25 | Ru(OAc)₂((S)- | CH₂Cl₂ | NEt₃ | 65 | 73 | (+) | 91.6 |
| | | | (6-MeO-2- | | | | | | |
| | | | Naphtyl)- | | | | | | |
| | | | MeOBIPHEP) | | | | | | |
| 11 | 0.05 | 25 | Ru(OAc)₂((S)- | AcOEt | NEt₃ | 65 | 80 | (+) | 89.0 |
| | | | (6-MeO-2- | | | | | | |
| | | | Naphtyl)- | | | | | | |
| | | | MeOBIPHEP) | | | | | | |
| 12 | 0.05 | 25 | Ru(OAc)₂((S)- | THF | NEt₃ | 65 | 78 | (+) | 79.6 |
| | | | (6-MeO-2- | | | | | | |
| | | | Naphtyl)- | | | | | | |
| | | | MeOBIPHEP) | | | | | | |
| 13 | 0.05 | 25 | Ru(OAc)₂((S)- | TFE | NEt₃ | 46 | 90 | (+) | 94.8 |
| | | | (6-MeO-2- | | | | | | |
| | | | Naphtyl)- | | | | | | |
| | | | MeOBIPHEP) | | | | | | |
| 14 | 0.05 | 25 | Ru(OAc)₂((S)- | MeOH/ | NEt₃ | 46 | 98 | (+) | 96.5 |
| | | | (6-MeO-2- | H₂O | | | | | |
| | | | Naphtyl)- | (9:1) | | | | | |
| | | | MeOBIPHEP) | | | | | | |

In a similar manner, but at different temperatures, different reaction times and under various pressure of hydrogen, the reactions in Table 6.1 were performed. Scale: 50 mg, S/C=25

TABLE 6.1

| Reaction | | | Base | | T | p | Yield | Major | e.e. |
|---|---|---|---|---|---|---|---|---|---|
| No. | Catalyst | Solvent | 1 equiv. | t (h) | (° C.) | (bar) | (%) | enantiomer | (%) |
| 1^{a} | Ru(OAc)₂((S)- | MeOH | NEt₃ | 64 | 60 | 40 | 88 | (+) | 96.9 |
| | (6-MeO-2- | | | | | | | | |
| | Naphtyl)- | | | | | | | | |
| | MeOBIPHEP) | | | | | | | | |
| 2^{a} | Ru(OAc)₂((S)- | MeOH | NEt₃ | 48 | 50 | 50 | >99 | (+) | 97.1 |
| | (6-MeO-2- | | | | | | | | |
| | Naphtyl)- | | | | | | | | |
| | MeOBIPHEP) | | | | | | | | |
| 3^{a} | Ru(OAc)₂((S)- | MeOH | NEt₃ | 44 | 40 | 40 | 82 | (+) | 96.9 |
| | (6-MeO-2- | | | | | | | | |
| | Naphtyl)- | | | | | | | | |
| | MeOBIPHEP) | | | | | | | | |
| 4^{a} | Ru(OAc)₂((S)- | MeOH | NEt₃ | 70 | Rt | 40 | 76 | (+) | 98.4 |
| | (6-MeO-2- | | | | (24–26° C.) | | | | |
| | Naphtyl)- | | | | | | | | |
| | MeOBIPHEP) | | | | | | | | |
| 5^{a} | Ru(OAc)₂((R)- | MeOH | NEt₃ | 24 | 80 | 40 | 78 | (−) | 96.7 |
| | (2-Furyl)- | | | | | | | | |
| | MeOBIPHEP) | | | | | | | | |
| 6^{a,b} | Ru(OAc)₂((R)- | MeOH | NEt₃ | 68 | 80 | 40 | 94 | (−) | 97.5 |
| | (2-Furyl)- | | | | | | | | |
| | MeOBIPHEP) | | | | | | | | |

^{a}35 ml autoclave,
^{b}Technical MeOH and NEt₃, autoclave loaded under air.

### EXAMPLE 7 OF I

### (+)-4-Phenyl-piperidine-1,3-dicarboxylic acid 1-tert butyl ester and (−)-4-Phenyl-piperidine-1,3-dicarboxylic acid 1-tert butyl ester

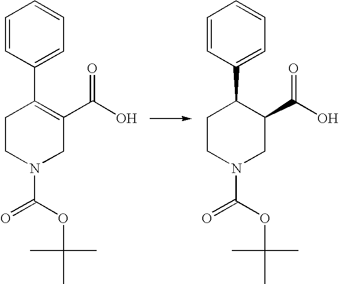

In a glove box (O₂ content≦2 ppm) a 6 ml autoclave equipped with a glass insert and a magnetic stirring bar was charged with 2.16 mg (0.0066 mmol) [Ru(OAc)₂(COD)], 6.71 mg (R_{C},Sₚ₁,S_{P})-TOLFER Stylacat 4/2 (0.00725 mmol) and methanol (1 ml). The corresponding catalyst solution was heated at 60° C. overnight (18 h in total), cooled to ambient temperature and charged with 0.05 g (0.165 mmol) 4-phenyl-5,6-dihydro-2H-pyridine-1,3-dicarboxylic acid-1-tert-butyl ester and 16.7 mg (0.165 mmol, 1 equiv.) of triethylamine. The asymmetric hydrogenation was run for 66 h at 80° C. under 40 bar of hydrogen. After cooling to room temperature the pressure was released from the autoclave, the methanol solution was diluted with 30 ml of tert-butyl methyl ether and extracted with two 30-ml portions of a 1 M aqueous sodium hydroxide solution. The aqueous layer was poured on ice, acidified with ice-cold 2 M aqueous hydrochloric acid solution to pH 1 and extracted with two 50-ml portions of ethyl acetate. The combined organic layers were dried over sodium sulfate, filtered and concentrated in vacuo to give (+)-(4-phenyl)-piperidine-1,3-dicarboxylic acid-1-tert-butyl ester in 91% yield (0.046 g) and with 97.3% ee.

MS m/e (%): 306 (M+H⁺, 100%).

### GC Method for ee Determination:

A 2-mg sample of the title compound was converted to the methyl ester by treatment with 0.5 ml of an approximately 0.5 M solution of diazomethane in diethyl ether at room temperature. After evaporation of excess diazomethane and diethyl ether under a gentle stream of argon the residue was dissolved in 1 ml of ethyl acetate. BGB-172 column, 30 m*0.25 mm*df 0.25 μm, hydrogen 150 kPa, split ratio 1:20; temperature gradient 180-230° C., program with 2° C./min; injector temperature 210° C., detector temperature 240° C. Retention times: 19.90 min (methyl ester of (+)-acid), 20.23 min (methyl ester of (−)-acid).

In analogy to the above described experiment, but with different chiral ligands, the reactions in Table 7 were performed.

TABLE 7

| Reaction | | Ruthenium | | t | Yield | Major | |
|---|---|---|---|---|---|---|---|
| No. | S/C | Precursor | Chiral Ligand | (h) | (%) | enantiomer | e.e. (%) |
| 1 | 25 | Ru(OAc)₂(COD) | (1R,1′R,2S,2′S)- | 68 | 99 | (−) | 82.6 |
| | | | DuanPhos | | | | |
| 2 | 25 | Ru(OAc)₂(COD) | (S_{c},Rₚₗ,Rₚ)-Stylacat 4/1 | 68 | 92 | (+) | 92.4 |
| 3 | 25 | Ru(OAc)₂(COD) | (S_{c},Rₚₗ,Rₚ)-Stylacat | 68 | 91 | (−) | 35.9 |
| | | | 3/1/1 | | | | |
| 4 | 25 | Ru(OAc)₂(COD) | (S)-f-BINAPHANE | 68 | 99 | (+) | 41.4 |
| 5 | 25 | Ru(OAc)₂(COD) | (S,S)-DIOP | 68 | 99 | (+) | 26.2 |
| 6 | 25 | Ru(OAc)₂(COD) | (R)—C2-Tunaphos | 68 | 99 | (−) | 93.1 |

### EXAMPLE 8 OF I

### (−)-2-Phenyl-cyclohexane carboxylic acid and (+)-2-Phenyl-cyclohexane carboxylic acid

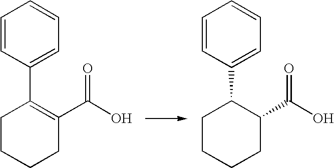

In a glove box (O₂ content≦2 ppm) a 6 ml autoclave equipped with a glass insert and a magnetic stirring bar was charged with 50 mg (0.25 mmol) of 2-phenyl-cyclohex-1-ene-carboxylic acid, 11.1 mg (0.00989 mmol) of [Ru(OAc)₂((R)-2-furyl)-MeOBIPHEP], 24.9 mg (0.247 mmol, 1.0 eq.) of triethylamine and 1 ml of methanol. The asymmetric hydrogenation was run for 42 h at 80° C. under 40 bar of hydrogen. After cooling to room temperature the pressure was released from the autoclave, the methanol solution was diluted with 30 ml of tert-butyl methyl ether and extracted with two 30-ml portions of a 1 M aqueous sodium hydroxide solution. The aqueous layer was poured on ice, acidified with ice-cold 2 M aqueous hydrochloric acid solution to pH 1 and extracted with two 50-ml portions of ethyl acetate. The combined organic layers were dried over sodium sulfate, filtered and concentrated in vacuo to give (−)-2-phenyl-cyclohexane carboxylic acid in 100% yield and with 95.1% ee.

MS m/e (%): 203 (M−H⁺, 100).

[α]_{D}=−76.42 (c=0.254, CHCl₃)

### HPLC Method for ee Determination:

Chiralpak-IA column, 25 cm*4.6 mm, 95% n-heptane+5% isopropanol with 1% trifluroacetic acid, flow 0.8 ml/min, 20° C., 0.002 ml injection volume, 215 nm. Retention times: (+)-acid 7.6 min, (−)-acid 8.2 min.

The reactions in Table 8 were performed according to the procedure above.

TABLE 8

| Reaction | Scale | | | NEt₃ | | Yield | Major | e.e. |
|---|---|---|---|---|---|---|---|---|
| No. | (g) | S/C | Catalyst | (equiv.) | t (h) | (%) | enantiomer | (%) |
| 1 | 0.05 | 25 | Ru(OAc)₂((rac)- | 1 | 42 | 79 | racemate | — |
| | | | BIPHEMP) | | | | | |
| 2 | 0.05 | 25 | Ru(OAc)₂((S)- | 1 | 42 | 100 | (+) | 90.8 |
| | | | 3,5-Xyl,4-MeO- | | | | | |
| | | | MeOBIPHEP) | | | | | |
| 3 | 0.05 | 25 | Ru(OAc)₂((S)-(6- | 1 | 42 | 100 | (+) | 90.4 |
| | | | MeO-2-Naphtyl)- | | | | | |
| | | | MeOBIPHEP) | | | | | |

### EXAMPLE 9 OF I

### (−)-2-Phenyl-cyclopentenecarboxylic acid and (+)-2-Phenyl-cyclopentenecarboxylic acid

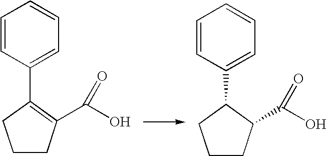

In a glove box (O₂ content≦2 ppm) a 6 ml autoclave equipped with a glass insert and a magnetic stirring bar was charged with 50 mg (0.27 mmol) of 2-phenyl-cyclopent-1-enecarboxylic acid, 8.1 mg (0.011 mmol) of [Ru(OAc)₂((R)-(2-furyl)-MeOBIPHEP], 26.8 mg (0.266 mmol, 1.0 eq.) of triethylamine and 1 ml of methanol. The asymmetric hydrogenation was run for 68 h at 80° C. under 40 bar of hydrogen. After cooling to room temperature the pressure was released from the autoclave, the methanol solution was diluted with 30 ml of tert-butyl methyl ether and extracted with two 30-ml portions of a 1 M aqueous sodium hydroxide solution. The aqueous layer was poured on ice, acidified with ice-cold 2 M aqueous hydrochloric acid solution to pH 1 and extracted with two 50-ml portions of ethyl acetate. The combined organic layers were dried over sodium sulfate, filtered and concentrated in vacuo to give (−)-2-phenyl-cyclopentenecarboxylic acid in 98% yield and with 97.1% ee.

MS m/e (%): 189 (M−H⁺, 100).

[α]_{D}=−85.22 (c=0.277, CHCl₃)

### HPLC Method for ee Determination:

Chiralpak-IA column, 25 cm*4.6 mm, 93% n-heptane+7% isopropanol with 1% trifluroacetic acid, flow 0.8 ml/min, 20° C., 0.002 ml injection volume, 215 nm. Retention times: (+)-acid 7.2 min, (−)-acid 7.8 min.

The reactions in Table 9 were performed according to the procedure above.

TABLE 9

| Reaction | Scale | | | Net₃ | | Yield | Major | e.e. |
|---|---|---|---|---|---|---|---|---|
| No. | (g) | S/C | Catalyst | (equiv.) | t (h) | (%) | enantiomer | (%) |
| 1 | 0.05 | 25 | Ru(OAc)₂((rac)- | 1 | 66 | 89 | racemate | — |
| | | | BIPHEMP) | | | | | |
| 2 | 0.05 | 25 | Ru(OAc)₂((S)3,5- | 1 | 68 | 96 | (+) | 78.6 |
| | | | Xyl,4-MeO- | | | | | |
| | | | MeOBIPHEP) | | | | | |
| 3 | 0.05 | 25 | Ru(OAc)₂((S)-(6- | 1 | 68 | 98 | (+) | 79.3 |
| | | | MeO-2-Naphtyl)- | | | | | |
| | | | MeOBIPHEP) | | | | | |

### EXAMPLE 10 OF I

### (+)-(3R,4R)-4-(Phenyl)-pyrrolidine-1,3-dicarboxylic acid-1-tert-butyl ester and (−)-(3S,4S)-4-(Phenyl)-pyrrolidine-1,3-dicarboxylic acid-1-tert-butyl ester

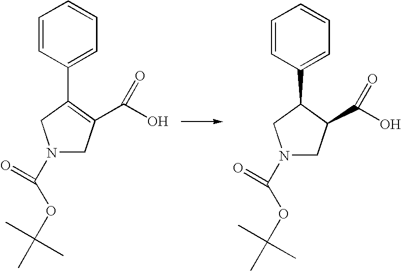

In a glove box (O₂ content≦2 ppm) a 185 ml autoclave equipped with a mechanical stirrer was charged with 4.46 g (15.4 mmol) of 4-(phenyl)-2,5-dihydro-pyrrole-1,3-dicarboxylic acid-1-tert-butyl ester, 173 mg (0.154 mmol) of [Ru(OAc)₂((S)-6-MeO-2-naphthyl)-MeOBIPHEP], 771 mg (7.62 mmol, 0.5 eq.) of triethylamine and 50 ml of methanol. The asymmetric hydrogenation was run for 48 h at 80° C. under 40 bar of hydrogen. After cooling to room temperature the pressure was released from the autoclave, the methanol solution was diluted with 200 ml of tert-butyl methyl ether and extracted with two 200-ml portions of a 1 M aqueous sodium hydroxide solution. The aqueous layer was poured on ice, acidified with ice-cold 2 M aqueous hydrochloric acid solution to pH 1 and extracted with three 300-ml portions of ethyl acetate. The combined organic layers were dried over sodium sulfate, filtered and concentrated in vacuo to give 3.95 g (88%) (+)-(3R,4R)-4-(phenyl)-pyrrolidine-1,3-dicarboxylic acid-1-tert-butyl ester with 90.5% ee. Crystallization from cyclohexane/ethyl acetate 9:1 gave 2.80 g (+)-(3R,4R)-4-(phenyl)-pyrrolidine-1,3-dicarboxylic acid-1-tert-butyl ester with 98.4% ee.

MS m/e (%): 290 (M−H⁺, 100).

[α]_{D}=+51.71 (c=0.700, CHCl₃)

### HPLC Method for ee Determination:

A 1-mg sample of the title compound was converted to the methyl ester by treatment with 0.5 ml of an approximately 0.5 M solution of diazomethane in diethyl ether at room temperature. After evaporation of excess diazomethane and diethyl ether under a gentle stream of argon the residue was dissolved in 1 ml of ethanol. Chiralpak-ADH column, 25 cm*4.6 mm, 93% n-heptane+7% ethanol, flow 0.7 ml/min, 25° C., 0.005 ml injection volume, 210 nm. Retention times: 11.3 min (methyl ester of (−)-acid), 14.6 min (methyl ester of (+)-acid).

### Assignment of the Absolute Configuration

To a solution of (+)-(3R,4R)-4-(phenyl)-pyrrolidine-1,3-dicarboxylic acid-1-tert-butyl ester (300 mg, 1.03 mmol, 98.4% ee) and triethylamine (167 mg, 1.65 mmol) in 10 ml tetrahydrofuran was added isobutyl chloroformate (211 mg, 1.54 mmol) at −10° C. After 30 minutes a solution of 2-mercaptopyridine N-oxide (275 mg, 2.16 mmol) and triethylamine (223 mg, 2.20 mmol) in 6 ml tetrahydrofuran was added. After completed addition the reaction mixture was warmed to room temperature and stirred for 3 h in the dark. After filtration and washing with 15 ml tetrahydrofuran 2-methyl-1-propanethiol (1.02 g, 11.3 mmol) the mixture was stirred under irradiation with a high-pressure mercury lamp for 20 h. After quenching with 2 M aqueous sodium hydroxide solution the mixture was extracted with three portions of tert-butyl methyl ether. The combined organic extracts were washed with brine, dried over sodium sulfate and concentrated in vacuo. The residue was purified by Kugelrohr distillation in high vacuo to give 206 mg (81%) (R)-3-phenyl-pyrrolidine-1-carboxylic acid tert-butyl ester.

MS m/e (%): 248 (M+H⁺, 10).

[α]_{D}=+13.52 (c=0.192, dichloromethane)

Lit.: A. I. Meyers, L. Snyder, J. Org. Chem. 1993, 58, 36. [α]_{D}=+10.3 (c=1.03, dichloromethane)

A solution of (R)-3-phenyl-pyrrolidine-1-carboxylic acid tert-butyl ester (140 mg, 0.566 mmol) in 4.5 ml of a 1.25 M solution of hydrochloric acid in methanol was stirred at 40° C. for 2 h. After evaporation of the solvent the residue was dissolved in a mixture of tert-butyl methyl ether and 2 M aqueous sodium hydroxide solution. The mixture was extracted with three portions of tert-butyl methyl ether. The combined organic extracts were dried over sodium sulfate and concentrated in vacuo. The residue was purified by Kugelrohr distillation in high vacuo to give 51 mg (61%) of (R)-3-phenyl-pyrrolidine.

MS m/e (%): 148 (M+H⁺, 100).

[α]_{D}=−22.32 (c=0.408, EtOH)

Lit.: C. C. Tseng et al. Chem. Pharm. Bull. 1977, 25, 166. For the (S) enantiomer

[α]_{D}=+22.7 (c=2.36, EtOH)

In a similar manner, but in a 6 ml or 35 ml autoclave, the reactions in Table 10 were performed.

TABLE 10

| Reaction | Scale | | | Et₃N | t | Yield | Major | e.e. |
|---|---|---|---|---|---|---|---|---|
| No. | (g) | S/C | Catalyst | (equiv.) | (h) | (%) | enantiomer | (%) |
| 1^{a)} | 0.2 | 25 | Ru(OAc)₂((R)- | 0.5 | 42 | 41 | (−) | 68 |
| | | | MeOBIPHEP) + 0.86 | | | | | |
| | | | toluene | | | | | |
| 2^{b)} | 0.05 | 25 | Ru(OAc)₂((R)- | 0.6 | 42 | 91 | (−) | 84.3 |
| | | | MeOBIPHEP) + 0.86 | | | | | |
| | | | toluene | | | | | |
| 3^{b)} | 0.05 | 25 | Ru(OAc)₂((S)-(6- | 0.6 | 42 | 99 | (+) | 94.9 |
| | | | MeO-2-Naphtyl)- | | | | | |
| | | | MeOBIPHEP) | | | | | |
| 4^{b)} | 0.05 | 25 | Ru(OAc)₂((R)- | 0.6 | 42 | 95 | (−) | 41.8 |
| | | | 3,5-tBu- | | | | | |
| | | | MeOBIPHEP) | | | | | |
| 5^{b)} | 0.05 | 25 | Ru(OAc)₂((+)- | 0.6 | 42 | >90 | (+) | 76.8 |
| | | | (S)-TMBTP | | | | | |
| 6^{b)} | 0.05 | 25 | Ru(OAc)₂((S)- | 0.6 | 42 | >90 | (+) | 94.0 |
| | | | 3,5-Xyl,4-MeO- | | | | | |
| | | | MeOBIPHEP) | | | | | |
| 7^{b)} | 0.05 | 25 | Ru (OAc)₂((all- | 0.6 | 42 | 89 | (+) | 71.5 |
| | | | S)-BICP) | | | | | |

^{a)}ml autoclave.
^{b)}ml autoclave.

### EXAMPLE 11 OF I

### (−)-4-(4-Chloro-phenyl)-pyrrolidine-1,3-dicarboxylic acid-1-tert-butyl ester and (+)-4-(4-Chloro-phenyl)-pyrrolidine-1,3-dicarboxylic acid-1-tert-butyl ester

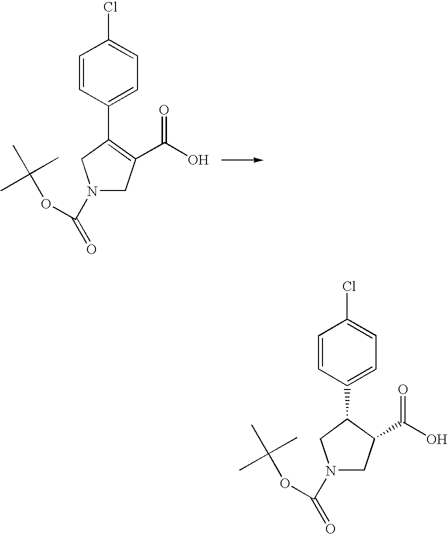

In a glove box (O₂ content≦2 ppm) a 6 ml autoclave equipped with a glass insert and a magnetic stirring bar was charged with 50 mg (0.154 mmol) of 4-(4-chloro-phenyl)-2,5-dihydro-pyrrole-1,3-dicarboxylic acid-1-tert-butyl ester, 4.7 mg (0.0062 mmol) of [Ru(OAc)₂((R)-2-furyl)-MeOBIPHEP], 15.4 mg (0.154 mmol, 1.0 eq.) of triethylamine and 1 ml of methanol. The asymmetric hydrogenation was run for 42 h at 80° C. under 40 bar of hydrogen. After cooling to room temperature the pressure was released from the autoclave, the methanol solution was diluted with 30 ml of tert-butyl methyl ether and extracted with two 30-ml portions of a 1 M aqueous sodium hydroxide solution. The aqueous layer was poured on ice, acidified with ice-cold 2 M aqueous hydrochloric acid solution to pH 1 and extracted with two 50-ml portions of ethyl acetate. The combined organic layers were dried over sodium sulfate, filtered and concentrated in vacuo to give (−)-4-(4-chloro-phenyl)-pyrrolidine-1,3-dicarboxylic acid-1-tert-butyl ester in 80% yield and with 98.3% ee.

MS m/e (%): 324 (M−H⁺, 100).

[α]_{D}=−50.37 (c=0.326, CHCl₃)

### HPLC Method for ee Determination:

Chiralpak-ADH column, 25 cm*4.6 mm, 85% n-heptane+15% ethanol with 0.5% trifluoroacetic acid, flow 0.7 ml/min, 20° C., 0.002 ml injection volume, 215 nm. Retention times: (+)-acid 10.6 min, (−)-acid 11.8 min.

The reactions in Table 11 were performed according to the procedure above.

TABLE 11

| Reaction | Scale | | | NEt₃ | | Yield | Major | e.e. |
|---|---|---|---|---|---|---|---|---|
| No. | (g) | S/C | Catalyst | (equiv.) | t (h) | (%) | enantiomer | (%) |
| 1 | 0.05 | 25 | Ru(OAc)₂((S)-(6-MeO- | 1 | 42 | 100 | (+) | 88.3 |
| | | | 2-Naphtyl)- | | | | | |
| | | | MeOBIPHEP) | | | | | |
| 2 | 0.05 | 25 | Ru(OAc)₂((S)3,5- | 1 | 42 | 80 | (+) | 85.7 |
| | | | Xyl,4-MeO- | | | | | |
| | | | MeOBIPHEP) | | | | | |

### EXAMPLE 12 OF I

### (+)-4-(3-Fluoro-phenyl)-pyrrolidine-1,3-dicarboxylic acid-1-tert-butyl ester and (−)-4-(3-Fluoro-phenyl)-pyrrolidine-1,3-dicarboxylic acid-1-tert-butyl ester

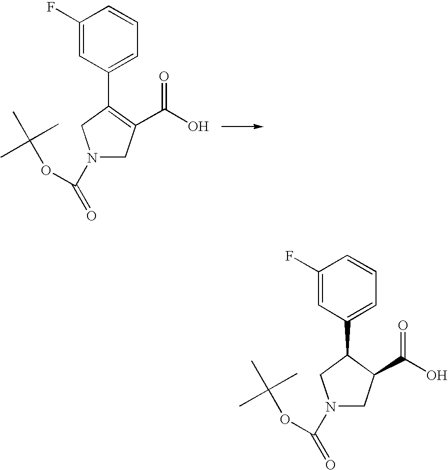

In a glove box (O₂ content≦2 ppm) a 6 ml autoclave equipped with a glass insert and a magnetic stirring bar was charged with 50 mg (0.16 mmol) of 4-(3-fluoro-phenyl)-2,5-dihydro-pyrrole-1,3-dicarboxylic acid-1-tert-butyl ester, 7.4 mg (0.0065 mmol) of [Ru(Oac)₂((S)-6-MeO-2-naphthyl)-MeOBIPHEP], 16.4 mg (0.163 mmol, 1.0 eq.) of triethylamine and 1 ml of methanol. The asymmetric hydrogenation was run for 42 h at 80° C. under 40 bar of hydrogen. After cooling to room temperature the pressure was released from the autoclave, the methanol solution was diluted with 30 ml of tert-butyl methyl ether and extracted with two 30-ml portions of a 1 M aqueous sodium hydroxide solution. The aqueous layer was poured on ice, acidified with ice-cold 2 M aqueous hydrochloric acid solution to pH 1 and extracted with two 50-ml portions of ethyl acetate. The combined organic layers were dried over sodium-sulphate, filtered and concentrated in vacuo to give (+)-4-(3-fluoro-phenyl)-pyrrolidine-1,3-dicarboxylic acid-1-tert-butyl ester in 77% yield and with 87.1% ee.

MS m/e (%): 308 (M−H⁺, 100).

### HPLC Method for ee Determination:

Chiralpak-ADH column, 25 cm*4.6 mm, 85% n-heptane+15% ethanol with 0.5% trifluoroacetic acid, flow 0.7 ml/min, 20° C., 0.002 ml injection volume, 215 nm. Retention times: (−)-acid 9.3 min, (+)-acid 11.2 min.

The reaction in Table 12 was performed according to the procedure above.

TABLE 12

| Reaction | Scale | | | NEt₃ | | Yield | Major | |
|---|---|---|---|---|---|---|---|---|
| No. | (g) | S/C | Catalyst | (equiv.) | t (h) | (%) | enantiomer | e.e. (%) |
| 1 | 0.025 | 25 | Ru(OAc)₂((R)-(2- | 1 | 42 | 100 | (−)^{a)} | 98.1 |
| | | | Furyl)- | | | | | |
| | | | MeOBIPHEP | | | | | |

^{a)}[α]_{D} = −46.03 (c = 0.341, CHCl₃).

### EXAMPLE 13 OF I

### (3R,4R)-1-Benzyl-4-phenyl-pyrrolidine-3-carboxylic acid and (3RS,4RS)-1-benzyl-4-phenyl-pyrrolidine-3-carboxylic acid

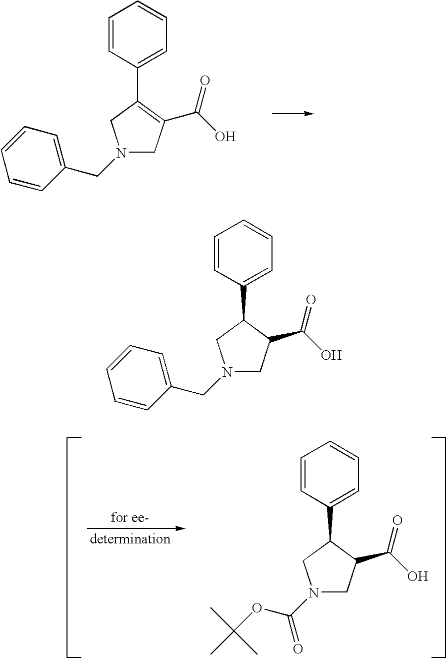

### Preparation of the Racemate:

In a glove box (O₂ content≦2 ppm) a 6 ml autoclave equipped with a glass insert and a magnetic stirring bar was charged with 50 mg (0.18 mmol) of 1-benzyl-4-phenyl-2,5-dihydro-1H-pyrrole-3-carboxylic acid, 5.5 mg (0.0072 mmol) of Ru(OAc)₂((rac)-BIPHEMP), 17.9 mg (0.179 mmol, 1.0 eq.) of triethylamine and 1 ml of methanol. The racemic hydrogenation was run for 42 h at 80° C. under 40 bar of hydrogen. After cooling to room temperature the pressure was released from the autoclave. The reaction mixture was diluted with 30 ml of tert-butyl methyl ether and extracted with two portions of a 1 M aqueous sodium hydroxide solution. The layers were separated and the aqueous phase was poured on ice. The pH was adjusted to pH 6 using 2 M aqueous hydrochloric acid solution. After extraction with three portions of dichloromethane (3×50 ml) the combined organic layers were dried over sodium sulphate, filtered and concentrated in vacuo to give (3RS,4RS)-1-benzyl-4-phenyl-pyrrolidine-3-carboxylic acid in 40% yield (20 mg).

MS m/e (%): 280 (M−H⁺, 100).

### Enantioselective Hydrogenation:

In a glove box (O₂ content≦2 ppm) a 6 ml autoclave equipped with a glass insert and a magnetic stirring bar was charged with 50 mg (0.18 mmol) of 1-benzyl-4-phenyl-2,5-dihydro-1H-pyrrole-3-carboxylic acid, 8.0 mg (0.0072 mmol) of [Ru(OAc)₂((S)-6-MeO-2-naphthyl)-MeOBIPHEP], 17.9 mg (0.179 mmol, 1.0 eq.) of triethylamine and 1 ml of methanol. The asymmetric hydrogenation was run for 68 h at 80° C. under 40 bar of hydrogen. After cooling to room temperature the pressure was released from the autoclave and the solvent was evaporated in vacuo. The residue was redissolved in 2 ml ethanol and 0.050 ml triethylamine (0.355 mmol) and 43 mg (0.20 mmol) di-tert-butyl dicarbonate were added. The reaction mixture was purged with argon prior the addition of Pd/C (10%) and then filled with hydrogen. The reaction mixture was stirred for 16 h at room temperature under hydrogen atmosphere and then filtered through Decalite. The filtrate was diluted with 30 ml of tert-butyl methyl ether and extracted with two portions of a 1 M aqueous sodium hydroxide solution. The layers were separated and the aqueous phase was poured on ice. The pH was adjusted to pH 1 using 2 M aqueous hydrochloric acid solution. After extraction with three portions of dichloromethane (3×50 ml) the combined organic layers were dried over sodium-sulphate, filtered and concentrated in vacuo to give (+)-(3R,4R)-4-(phenyl)-pyrrolidine-1,3-dicarboxylic acid-1-tert-butyl ester in 6% yield and with 97.4% ee.

MS m/e (%): 290 (M−H⁺, 100).

### HPLC Method for ee Determination:

Chiralpak-ADH column, 25 cm*4.6 mm, 93% n-heptane+7% ethanol, flow 0.7 ml/min, 25° C., 0.003 ml injection volume, 210 nm. Retention times: 11.3 min (methyl ester of (−)-acid), 14.6 min (methyl ester of (+)-acid).

The reactions in Table 13 were performed according to the procedure above.

TABLE 13

| Reaction | Scale | | | NEt₃ | | Yield | Major | e.e. |
|---|---|---|---|---|---|---|---|---|
| No. | (g) | S/C | Catalyst | (equiv.) | t (h) | (%) | enantiomer^{a)} | (%)^{a)} |
| 1 | 0.05 | 25 | Ru(OAc)₂((rac)- | 1 | 42 | 40 | racemate | — |
| | | | BIPHEMP) | | | | | |
| 2 | 0.05 | 25 | Ru(OAc)₂((S)3,5- | 1 | 68 | 23 | (+) | 97.7 |
| | | | Xyl,4-MeO- | | | | | |
| | | | MeOBIPHEP) | | | | | |
| 3 | 0.05 | 25 | Ru(OAc)₂((S)- | 1 | 68 | 25 | (+) | 92.7 |
| | | | BITIANP) | | | | | |

^{a)}Optical rotation and ee of (+)-(3R,4R)- or (−)-(3S,4S)-4-(phenyl)-pyrrolidine-1,3-di carboxylic acid-1-tert-butyl ester obtained after debenzylation and N-tert-butoxycarbonyl protection of the primary hydrogenation product (3R,4R)- or (3S,4S)-1-benzyl-4-phenyl-pyrrolidine-3-carboxylic acid.

### EXAMPLE 14 OF 1

### (+)-4-Phenyl-tetrahydro-thiophene-3-carboxylic acid and (−)-4-Phenyl-tetrahydro-thiophene-3-carboxylic acid

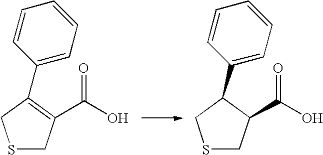

In a glove box (O₂ content≦2 ppm) a 6 ml autoclave equipped with a glass insert and a magnetic stirring bar was charged with 0.050 g (0.242 mmol) of 4-phenyl-2,5-dihydrothiophene-3-carboxylic acid, 36.92 mg (0.0485 mmol) of [Ru(OAc)₂((R)-2-Furyl)-MeOBIPHEP], 24.5 mg (0.242 mmol, 1 eq.) of triethylamine and 1 ml of methanol. The asymmetric hydrogenation was run for 70 h at 80° C. under 40 bar of hydrogen. After cooling to room temperature the pressure was released from the autoclave, the methanol solution was diluted with 30 ml of tert-butyl methyl ether and extracted with two 30-ml portions of a 1 M aqueous sodium hydroxide solution. The aqueous layer was poured on ice, acidified with ice-cold 2 M aqueous hydrochloric acid solution to pH 1 and extracted with two 50-ml portions of ethyl acetate. The combined organic layers were dried over sodium sulfate, filtered and concentrated in vacuo to give (+)-4-phenyl-tetrahydro-thiophene-3-carboxylic acid in 60% yield (0.03 g) and with 98.1% ee.

MS m/e (%): 207 (M+−H, 100).

[α]_{D}=+33.93 (c=0.342, CHCl₃)

### GC Method for ee Determination:

A 2-mg sample of the title compound was converted to the methyl ester by treatment with 0.5 ml of an approximately 0.5 M solution of diazomethane in diethyl ether at room temperature. After evaporation of excess diazomethane and diethyl ether under a gentle stream of argon the residue was dissolved in 1 ml of ethyl acetate. BGB-172 column, 60 m*0.25 mm*df 0.25 μm, hydrogen 150 kPa, split ratio 1:50; temperature gradient 160-230° C., program with 2° C./min; injector temperature 210° C., detector temperature 230° C. Retention times: 33.11 min (methyl ester of (+)-acid), 33.57 min (methyl ester of (−)-acid).

The reactions in Table 14 were performed according to the procedure above.

TABLE 14

| Reaction | Scale | | | | Base (1 | | Yield | Major | |
|---|---|---|---|---|---|---|---|---|---|
| No. | (g) | S/C | Catalyst | Solvent | equiv.) | t (h) | (%) | enantiomer | e.e. (%) |
| 1 | 0.05 | 5 | Ru(OAc)₂((S)- | MeOH | NEt₃ | 68 | 99 | (−) | 73.0 |
| | | | (6-MeO-2- | | | | | | |
| | | | Naphtyl)- | | | | | | |
| | | | MeOBIPHEP) | | | | | | |
| 2 | 0.05 | 5 | Ru(OAc)₂((S)- | MeOH | NEt₃ | 68 | 58 | (−) | 74.5 |
| | | | (BITIANP) | | | | | | |

### EXAMPLE 15 OF I

### (−)-2-Phenyl-cyclooctanecarboxylic acid

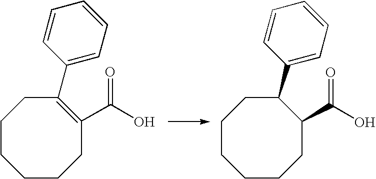

In a glove box (O₂ content≦2 ppm) a 35 ml autoclave equipped with a 15 ml glass insert and a magnetic stirring bar was charged with 0.050 g (0.217 mmol) of 2-phenyl-cyclooct-1-ene-carboxylic acid, 9.31 mg (0.00868 mmol) of [Ru((S)-MeOBIPHEP)(pCym)I]I, 2.2 mg (0.0217 mmol, 0.1 eq.) of triethylamine and 1 ml of methanol. The asymmetric hydrogenation was run for 42 h at 80° C. under 40 bar of hydrogen. After cooling to room temperature the pressure was released from the autoclave, the methanol solution was diluted with 30 ml of tert-butyl methyl ether and extracted with two 30-ml portions of a 1 M aqueous sodium hydroxide solution. The aqueous layer was poured on ice, acidified with ice-cold 2 M aqueous hydrochloric acid solution to pH 1 and extracted with two 50-ml portions of ethyl acetate. The combined organic layers were dried over sodium sulfate, filtered and concentrated in vacuo to give (−)-(2-phenyl)-cyclooctanecarboxylic acid in 76% yield (0.036 g) and with 45.9% ee.

MS m/e (%): 231 (M−H⁺, 100).

[α]_{D}=−3.97 (c=0.504, CHCl₃)

### GC Method for ee Determination:

A 2-mg sample of the title compound was converted to the methyl ester by treatment with 0.5 ml of an approximately 0.5 M solution of diazomethane in diethyl ether at room temperature. After evaporation of excess diazomethane and diethyl ether under a gentle stream of argon the residue was dissolved in 1 ml of ethyl acetate. BGB-172 column, 60 m*0.25 mm*df 0.25 μm, hydrogen 150 kPa, split ratio 1:50; temperature gradient 160-230° C., program with 2° C./min; injector temperature 210° C., detector temperature 230° C. Retention times: 32.66 min (methyl ester of (+)-acid), 32.85 min (methyl ester of (−)-acid).

In a similar manner, the reactions in Table 15 were performed.

TABLE 15

| | | | | | | | Conv. (%) | | |
|---|---|---|---|---|---|---|---|---|---|
| Reaction | Scale | | | | Base (1 | t | (isol. Yield | Major | e.e. |
| No. | (g) | S/C | Catalyst | Solvent | equiv.) | (h) | (%)) | enantiomer | (%) |
| 1 | 0.05 | 25 | Ru(OAc)₂((S)-(6- | MeOH | NEt₃ | 67 | >99 (80) | (−) | 34.6 |
| | | | MeO-2-Naphtyl)- | | | | | | |
| | | | MeOBIPHEP) | | | | | | |
| 2 | 0.05 | 25 | Ru(OAc)₂((S)- | MeOH | NEt₃ | 67 | >99 (92) | (−) | 38.6 |
| | | | pTol- | | | | | | |
| | | | MeOBIPHEP | | | | | | |
| 3 | 0.05 | 25 | [Ru((S)- | MeOH | none | 67 | 55 (n.d.)^{a} | (−) | 81.4 |
| | | | MeOBIPHEP)(pC | | | | | | |
| | | | ym)I]I | | | | | | |
| 4 | 0.05 | 6 | [Ru((S)-3,5-tBu- | MeOH | none | 20 | 17 (n.d.)^{a} | (−) | 95.8 |
| | | | MeOBIPHEP)(C6H6)Cl]BF4 | | | | | | |

^{a}Yield not determined

Synthesis of cyclic β-aryl substituted α,β-unsaturated carboxylic acids II as starting materials for the enantioselective hydrogenations:

### EXAMPLE 1 OF II

### 4-(4-Fluoro-phenyl)-5,6-dihydro-2H-pyridine-1,3-dicarboxylic acid 1-tert-butyl ester

### a) 4-Trifluoromethanesulfonyloxy-5,6-dihydro-2H-pyridine-1,3-dicarboxylic acid 1-tert-butyl ester 3-methyl ester

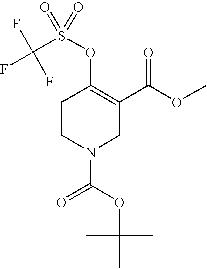

To a solution of 4-oxo-piperidine-1,3-dicarboxylic acid 1-tert-butyl ester 3-methyl ester (8.64 g, 33.5 mmol) in 230 ml THF was added sodium hydride (suspension in oil, 55%, 3.26 g, 74.6 mmol) at 0° C. After stirring for 30 min. at 0° C. N-phenyltrifluoromethanesulfonimide (20.4 g, 56.0 mmol) was added. The ice-water bath was removed and the reaction mixture was stirred for 2 days. Quenching with ice was followed by concentration in vacuo to remove THP. The residue was diluted with tert-butyl methyl ether and washed with three portions of 1 M aqueous sodium hydroxide solution. The organic layer was washed with brine and dried over sodium sulfate. Concentration in vacuo gave the crude title compound with a purity of 90% (11.4 g, 26.4 mmol, 71%).

MS m/e (%): 334 (M+H⁺—C₄H₈, 100).

### b) 4-(4-Fluoro-phenyl)-5,6-dihydro-2H-pyridine-1,3-dicarboxylic acid 1-tert-butyl ester 3-methyl ester

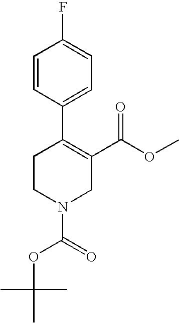

To a mixture of 4-trifluoromethanesulfonyloxy-5,6-dihydro-2H-pyridine-1,3-dicarboxylic acid 1-tert-butyl ester 3-methyl ester (10.1 g, 25.9 mmol), 4-fluorophenylzinc bromide solution (0.5 M in THF, 86.3 ml, 43.1 mmol) and 290 ml THF was added tetrakis(triphenylphosphine)palladium(0) (0.83 g, 0.72 mmol) at RT. After stirring for 6 h the reaction was quenched with ice. The mixture was diluted with tert-butyl methyl ether and washed with 2 M aqueous sodium carbonate solution. The aqueous layer was extracted with two portions of tert-butyl methyl ether. The combined organic layers were washed with brine, dried over sodium sulfate and concentrated in vacuo. Purification of the residue by flash chromatography (heptane/ethyl) gave the title compound as a lightly yellow amorphous residue (6.8 g, 71%).

MS m/e (%): 336 (M+H⁺, 10).

### c) 4-(4-Fluoro-phenyl)-5,6-dihydro-2H-pyridine-1,3-dicarboxylic acid 1-tert-butyl ester

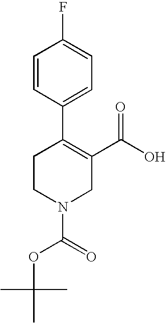

A mixture of 4-(4-fluoro-phenyl)-5,6-dihydro-2H-pyridine-1,3-dicarboxylic acid 1-tert-butyl ester 3-methyl ester (6.8 g, 20 mmol), 100 ml 1,4-dioxane and 100 ml 2 M NaOH was stirred at RT for 20 h. After extraction of the reaction mixture with two portions of tert-butyl methyl ether, the combined organic layers were extracted with 1 M aqueous sodium hydroxide solution (100 ml). The combined aqueous layers were cooled to 0° C. by addition of ice (150 g) and acidified to pH 1 with ice-cold 4 M aqueous hydrochloric acid solution (70 ml). The aqueous layer was extracted with three 150 ml-portions of ethyl acetate. The combined organic layers were washed with brine (50 ml), dried over sodium sulfate and concentrated in vacuo. Crystallization of the crude acid (6.4 g) from a mixture of n-heptane and ethyl acetate (19:1, 120 ml) gave the title compound as white crystals (5.1 g, 78%).

MS m/e (%): 320 (M−H⁺, 100).

### EXAMPLE 2 OF II

### 4-(1H-Indol-3-yl)-5,6-dihydro-2H-pyridine-1,3-dicarboxylic acid 1-tert-butyl ester

### a) 4-[1-(tert-Butyl-dimethyl-silanyl)-1H-indol-3-yl]-5,6-dihydro-2H-pyridine-1,3-dicarboxylic acid 1-tert-butyl ester 3-methyl ester

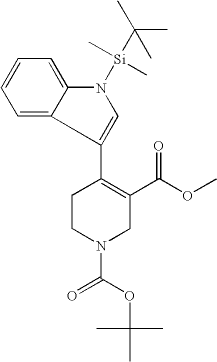

To a solution of 3-bromo-1-(tert-butyl-dimethyl-silanyl)-1H-indole (23.0 g, 74.1 mmol) in dry THF (280 ml) was added dropwise at −78° C. a solution of tert-butyllithium in pentane (1.7 M, 87.2 ml, 148 mmol). To the resulting orange solution was added dropwise a freshly prepare solution of dried zink chloride (11.1 g, 81.5 mmol) in dry THF (110 ml) at −78° C. After completed addition the reaction mixture was allowed to slowly warm to room temperature over a period of 1.5 h. To this mixture were added a solution of 4-trifluoromethanesulfonyloxy-5,6-dihydro-2H-pyridine-1,3-dicarboxylic acid 1-tert-butyl ester 3-methyl ester (19.6 g, 50.3 mmol) in THF (130 ml) and tetrakis(triphenylphosphine)palladium(0) (1.75 g, 1.51 mmol). After stirring for 64 h at room temperature the reaction was quenched with ice. The mixture was diluted with tert-butyl methyl ether and washed with 2 M aqueous sodium carbonate solution.

The aqueous layer was extracted with two portions of tert-butyl methyl ether. The combined organic layers were washed with water and brine, dried over sodium sulfate and concentrated in vacuo. Purification of the residue by flash chromatography (heptane/ethyl) gave the title compound as an amorphous residue (18.0 g, 76%).

MS m/e (%): 471 (M+H⁺, 85).

### b) 4-(1H-Indol-3-yl)-5,6-dihydro-2H-pyridine-1,3-dicarboxylic acid 1-tert-butyl ester

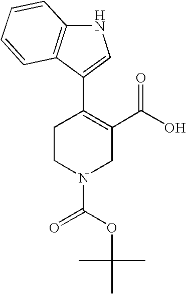

The title compound was obtained as a light brown solid after trituration from THF in comparable yield according to the procedure described above for the preparation of 4-(4-fluoro-phenyl)-5,6-dihydro-2H-pyridine-1,3-dicarboxylic acid 1-tert-butyl ester using 4-[1-(tert-butyl-dimethyl-silanyl)-1H-indol-3-yl]-5,6-dihydro-2H-pyridine-1,3-dicarboxylic acid 1-tert-butyl ester 3-methyl ester instead of 4-(4-fluoro-phenyl)-5,6-dihydro-2H-pyridine-1,3-dicarboxylic acid 1-tert-butyl ester 3-methyl ester in step c).

MS m/e (%): 341 (M−H⁺, 100)

### EXAMPLE 3 OF II

### 4-o-Tolyl-5,6-dihydro-2H-pyridine-1,3-dicarboxylic acid 1-tert-butyl ester

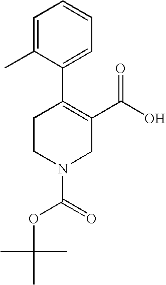

The title compound was obtained as white crystals in comparable yields according to the procedures described above for the preparation of 4-(1H-indol-3-yl)-5,6-dihydro-2H-pyridine-1,3-dicarboxylic acid 1-tert-butyl ester using o-tolylmagnesium chloride instead of 3-lithio-1-(tert-butyl-dimethyl-silanyl)-1H-indole freshly prepared from 3-bromo-1-(tert-butyl-dimethyl-silanyl)-1H-indole and tert-butyllithium in step a).

MS m/e (%): 316 (M−H⁺, 100)

### EXAMPLE 4 OF II

### 4-(3-Methoxy-phenyl)-5,6-dihydro-2H-pyridine-1,3-dicarboxylic acid 1-tert-butyl ester

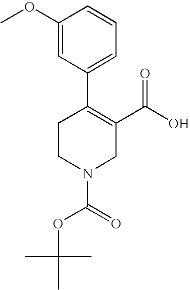

The title compound was obtained as off-white crystals in comparable yields according to the procedures described above for the preparation of 4-(4-fluoro-phenyl)-5,6-dihydro-2H-pyridine-1,3-dicarboxylic acid 1-tert-butyl ester using 3-methoxyphenylzinc iodide instead of 4-fluorophenylzinc bromide in step b).

MS m/e (%): 332 (M−H⁺, 100)

### EXAMPLE 5 OF II

### 4-Phenyl-5,6-dihydro-2H-pyridine-1,3-dicarboxylic acid 1-tert-butyl ester

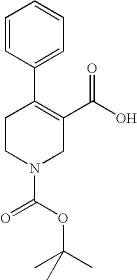

The title compound was obtained as off-white crystals in comparable yields according to the procedures described above for the preparation of 4-(4-fluoro-phenyl)-5,6-dihydro-2H-pyridine-1,3-dicarboxylic acid 1-tert-butyl ester using phenylzinc iodide instead of 4-fluorophenylzinc bromide in step b).

MS m/e (%): 302 (M−H⁺, 100)

### EXAMPLE 6 OF II

### 5-Phenyl-3,6-dihydro-2H-pyridine-1,4-dicarboxylic acid 1-tert-butyl ester

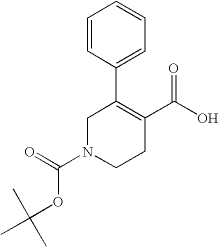

The title compound was obtained as a colorless viscous oil after flash column chromatography in comparable yields according to the procedures described above for the preparation of 4-(4-fluoro-phenyl)-5,6-dihydro-2H-pyridine-1,3-dicarboxylic acid 1-tert-butyl ester using 3-oxo-piperidine-1,4-dicarboxylic acid 1-tert-butyl ester 4-ethyl ester instead of 4-oxo-piperidine-1,3-dicarboxylic acid 1-tert-butyl ester 3-methyl ester in step a) and phenylzinc iodide instead of 4-fluorophenylzinc bromide in step b).

MS m/e (%): 302 (M−H⁺, 100)

### EXAMPLE 7 OF II

### 2-Phenyl-cyclohex-1-enecarboxylic acid

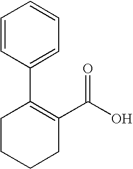

The title compound was obtained as white crystals in comparable yields according to the procedures described above for the preparation of 4-(4-fluoro-phenyl)-5,6-dihydro-2H-pyridine-1,3-dicarboxylic acid 1-tert-butyl ester using cyclohexanone-2-carboxylic acid ethylester instead of 4-oxo-piperidine-1,3-dicarboxylic acid 1-tert-butyl ester 3-methyl ester in step a) and phenylzinc iodide instead of 4-fluorophenylzinc bromide in step b).

MS m/e (%): 201 (M−H⁺, 100)

### EXAMPLE 8 OF II

### 2-Phenyl-cyclopent-1-enecarboxylic acid

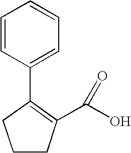

The title compound was obtained as off-white crystals in comparable yields according to the procedures described above for the preparation of 4-(4-fluoro-phenyl)-5,6-dihydro-2H-pyridine-1,3-dicarboxylic acid 1-tert-butyl ester using cyclopentanone-2-carboxylic acid methylester instead of 4-oxo-piperidine-1,3-dicarboxylic acid 1-tert-butyl ester 3-methyl ester in step a) and phenylzinc iodide instead of 4-fluorophenylzinc bromide in step b).

MS m/e (%): 187 (M−H⁺, 100)

### EXAMPLE 9 OF II

### 4-Phenyl-2,5-dihydro-pyrrole-1,3-dicarboxylic acid 1-tert-butyl ester

### a) 1-Benzyl-4-phenyl-2,5-dihydro-1H-pyrrole-3-carboxylic acid ethyl ester

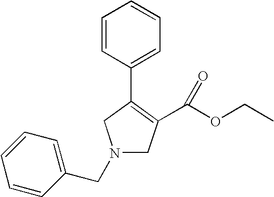

A solution of ethyl phenylpropiolate (12.0 g, 68.9 mmol) and N-(methoxymethyl)-N-(trimethylsilylmethyl) benzylamine (26.2 g, 110 mmol) in 180 ml dichloromethane was cooled to 0° C. with an ice-water bath. Trifluoroacetic acid (0.53 ml, 6.9 mmol) was added slowly, keeping the temperature of the reaction mixture below 20° C. After completed addition the mixture was stirred at room temperature for 16 h. The solvent was removed under reduced pressure. The residue was dissolved in 2 M aqueous hydrochloric acid solution (150 ml) and extracted with three portions of n-heptane (3×100 ml). The aqueous layer was basified with 32% aqueous sodium hydroxide solution (30 ml) and extracted with three portions of ethyl acetate (3×150 ml). The combined ethyl acetate extracts were washed with brine, dried over sodium sulfate and concentrated under reduced pressure. Flash chromatography (n-heptane/ethyl acetate) gave the title compound (17.0 g, 80%) as a slightly yellow oil.

MS m/e (%): 308.5 (M+H⁺, 100).

### b) 4-Phenyl-2,5-dihydro-pyrrole-1,3-dicarboxylic acid 1-tert-butyl ester

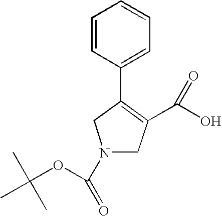

A mixture of 1-benzyl-4-phenyl-2,5-dihydro-1H-pyrrole-3-carboxylic acid ethyl ester (25.0 g, 81.3 mmol) and 1-chloroethyl chloroformate (10.7 ml, 97.6 mmol) in 450 ml 1,2-dichloroethane was heated at 50° C. for 24 h. After evaporation of the solvent the residue was dissolved in methanol and heated at reflux for 1 h. The reaction mixture was concentrated in vacuo and the residual hydrochloride was redissolved in a mixture of 450 ml THF and triethylamine (34.0 ml, 244 mmol). Di-tert-butyl dicarbonate (26.6 g, 122 mmol) was added at 0° C., and the reaction mixture was stirred for 1 h. The reaction mixture was diluted with saturated aqueous ammonium chloride solution and extracted with three portions of tert-butyl methyl ether (3×200 ml). The combined organic layers were dried over sodium sulfate and concentrated in vacuo to give 40 g of crude 4-phenyl-2,5-dihydro-pyrrole-1,3-dicarboxylic acid 1-tert-butyl ester 3-ethyl ester, which was contaminated mainly with di-tert-butyl dicarbonate and benzyl chloride, as a yellow oil. A mixture of this material, 400 ml 1,4-dioxane and 400 ml 2 M aqeuous sodium hydroxide solution was stirred at room temperature over night. The reaction mixture was washed with two portions of hepane. The aqueous layer was acidified with ice-cold 4 M aqueous hydrochloric acid solution (270 ml). Filtration and washing with cold water gave the title compound as white crystals (19.7 g, 83%).

MS m/e (%): 288 (M−H⁺, 100).

### EXAMPLE 10 OF II

### 4-(4-Chloro-phenyl)-2,5-dihydro-pyrrole-1,3-dicarboxylic acid 1-tert-butyl ester

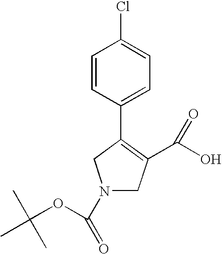

The title compound was obtained as white crystals according to the procedures described above for the preparation of 4-phenyl-2,5-dihydro-pyrrole-1,3-dicarboxylic acid 1-tert-butyl ester using methyl (4-chlorophenyl)propiolate (prepared as described by T. Eckert, J. Ipaktschi, Synthetic Communications 1998, 28, 327.) instead of ethyl phenylpropiolate in step a).

MS m/e (%): 268 (M+H⁺—C₄H₉, 100).

### EXAMPLE 11 OF II

### 4-(3-Fluoro-phenyl)-2,5-dihydro-pyrrole-1,3-dicarboxylic acid 1-tert-butyl ester

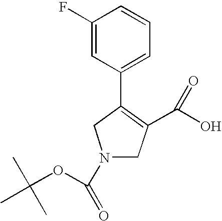

The title compound was obtained as off-white crystals according to the procedures described above for the preparation of 4-phenyl-2,5-dihydro-pyrrole-1,3-dicarboxylic acid 1-tert-butyl ester using methyl (3-fluorophenyl)propiolate (prepared as described by T. Eckert, J. Ipaktschi, Synthetic Communications 1998, 28, 327.) instead of ethyl phenylpropiolate in step a).

MS m/e (%): 306 (M−H⁺, 69).

### EXAMPLE 12 OF II

### 1-Benzyl-4-phenyl-2,5-dihydro-1H-pyrrole-3-carboxylic acid

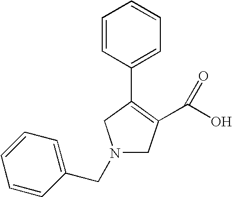

A mixture of 1-benzyl-4-phenyl-2,5-dihydro-1H-pyrrole-3-carboxylic acid ethyl ester (1.88 g, 6.12 mmol), 33 ml 1,4-dioxane and 33 ml 2 M aqeuous sodium hydroxide solution was stirred at room temperature over night. The mixture was acidified to pH 4 with ice-cold 4 M aqueous hydrochloric acid solution and extracted with three portions of dichloromethane. The combined organic layers were dried over sodium sulfate and concentrated in vacuo to give 1.2 g of a white solid. Trituration from warm ethanol and filtration gave the title compound (0.54 g, 32%) as a white solid.

MS m/e (%): 278 (M−H⁺, 100).

### EXAMPLE 13 OF II

### 4-Phenyl-2,5-dihydro-thiophene-3-carboxylic acid

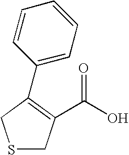

The title compound has been synthesized in comparable yields according to the following literature procedures using toluene instead of benzene for the formation of thio benzoic acid:
a) T. Aoyama, T. Takido, M. Kodomari, Synth. Comm. 2003, 33 (21), 3817;
b) D. H. Martyres, J. E. Baldwin, R. M. Adlington, V. Lee, M. R. Probert, D. J. Watkin, Tetrahedron 2001, 57, 4999;
c) G: M. Coppola, R. E. Damon, H. Xu, Synlett 1995, 11, 1143.

MS m/e (%): 205 (M−H⁺, 100).

### EXAMPLE 14 OF II

### 2-Phenyl-cyclooct-1-enecarboxylic acid

### a) 2-Trifluoromethanesulfonyloxy-1,2-dihydro-1-carboxylic acid ethyl ester

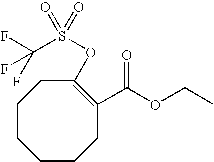

To a solution of 2-Oxo-cyclooctanecarboxylic acid ethyl ester (9.65 g, 47.2 mmol) in 33 ml THF was added sodium hydride (suspension in oil, 55%, 4.57 g, 104.8 mmol) at 0° C. After stirring for 30 min. at 0° C. N-phenyltrifluoromethanesulfonimide (28.17 g, 78.8 mmol) was added. The ice-water bath was removed and the reaction mixture was stirred for 2 days. Quenching with ice was followed by concentration in vacuo to remove THF. The residue was diluted with tert-butyl methyl ether and washed with three portions of 1 M aqueous sodium hydroxide solution. The organic layer was washed with brine and dried over sodium sulfate. Concentration in vacuo gave the crude title compound with a purity of 94% (15.41 g, 93%).

MS m/e (%): 285 ([M-OCH₂CH₃]⁺, 100).

### b) 2-Phenyl-cyclooct-1-enecarboxylic acid ethyl ester

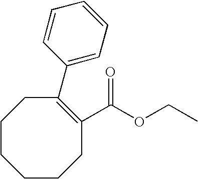

To a mixture of 2-Trifluoromethanesulfonyloxy-1,2-dihydro-1-carboxylic acid ethyl ester (10.35 g, 29.8 mmol), phenylzinc iodide solution (0.5 M in THF, 98.8 ml, 49.4 mmol) and 330 ml THF was added tetrakis(triphenylphosphine)palladium(0) (2.08 g, 1.78 mmol) and lithium chloride (1.27 g, 29.8 mmol) at RT. After stirring for 27 h the reaction was quenched with ice. The mixture was diluted with tert-butyl methyl ether and washed with 2 M aqueous sodium carbonate solution. The aqueous layer was extracted with two portions of tert-butyl methyl ether. The combined organic layers were washed with brine, dried over sodium sulfate and concentrated in vacuo. Purification of the residue by flash chromatography (heptane/ethyl acetate 50:1) gave the title compound as a colourless oil in 90% purity (4.57 g, 54%).

MS m/e (%): 259 (M+H⁺, 100%).

### c) 2-Phenyl-cyclooct-1-enecarboxylic acid

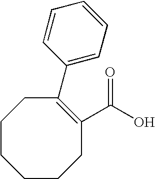

A mixture of 2-Phenyl-cyclooct-1-enecarboxylic acid ethyl ester (3.44 g, 9.02 mmol), 172 ml 1,4-dioxane and 172 ml 1 M LiOH was refluxed for 20 h. After cooling to ambient temperature and extraction of the reaction mixture with two portions of tert-butyl methyl ether (440 ml in total), the combined organic layers were extracted with 1 M aqueous sodium hydroxide solution (220 ml). The combined aqueous layers were cooled to 0° C. by addition of ice (150 g) and acidified to pH 1 with ice-cold 4 M aqueous hydrochloric acid solution (100 ml). The aqueous layer was extracted with two 250 ml-portions of ethyl acetate. The combined organic layers were washed with brine (50 ml), dried over sodium sulfate and concentrated in vacuo. Crystallization of the crude acid from a mixture of n-heptane and ethyl acetate (13:1, 210 ml) gave the title compound as off-white crystals (2.3 g, 75%).

MS m/e (%): 229 (M−H⁺, 100).

### Representative Procedure for the Epimerization of Enantiomerically Enriched cis-Substituted Cyclic β-arylcarboxcylic Acid Derivates

### EXAMPLE 1 OF III

### (+)-(3R,4R)-4-(4-Fluoro-phenyl)-piperidine-1,3-dicarboxylic acid 1-tert-butyl ester 3-methyl ester

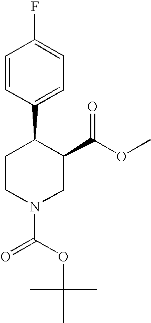

To a solution of triphenylphosphine (3.82 g, 14.6 mmol) in 70 ml tetrahydrofuran was added diethyl azodicarboxylate (2.53 g, 14.6 mmol) at 0° C. After 30′ methanol (4.55 ml, 112.0 mmol) and a solution of (3R,4R)-4-(4-fluoro-phenyl)-piperidine-1,3-dicarboxylic acid 1-tert-butyl ester (3.62 g, 11.2 mmol, 93.6% ee) in 30 ml tetrahydrofuran were added subsequently at 0-5° C. The reaction mixture was stirred for 20 h at room temperature. Quenching with water was followed by extraction with tert-butyl methyl ether (3×100 ml). The combined organic layers were dried over sodium sulfate, concentrated under reduced pressure and purified by flash chromatography (n-heptane/ethyl acetate) to give the title compound (3.55 g, 94%) as a colorless oil.

MS m/e (%): 338 (M+H⁺, 28).

[α]_{D}=+68.69 (c=0.310, CHCl₃)

[α]₅₇₈=+71.27 (c=0.310, CHCl₃)

[α]₃₆₅=+221.60 (c=0.310, CHCl₃)

### EXAMPLE 1 OF V

### (−)-(3S,4R)-4-(4-Fluoro-phenyl)-piperidine-1,3-dicarboxylic acid 1-tert-butyl ester

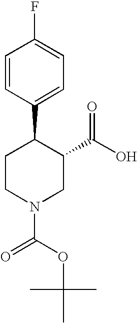

A mixture of (+)-(3R,4R)-4-(4-fluoro-phenyl)-piperidine-1,3-dicarboxylic acid 1-tert-butyl ester 3-methyl ester (3.55 g, 10.5 mmol) and sodium methoxide (1.14 g, 21.1 mmol) in 100 ml anhydrous toluene was heated at reflux over night. After cooling to room temperature the reaction mixture was quenched with water and concentrated in vacuo. The residue was dissolved in a mixture of 100 ml 1,4-dioxane and 50 ml 2 M aqueous sodium hydroxide solution. After stirring at RT for 5 h the mixture was diluted with water and washed with two portions of tert-butyl methyl ether. The aqueous layer was cooled to 0° C., acidified to pH 1-2 with ice-cold 1 M aqueous hydrochloric acid solution and extracted with three portions of tert-butyl methyl ether. The combined organic layers were dried over sodium sulfate and concentrated in vacuo. Flash column chromatography and crystallization from heptane/ethyl acetate 9:1 (30 ml) gave the title compound as white crystals (1.76 g, 52%, 97.5% ee).

MS m/e (%): 322 (M−H⁺, 100).

[α]_{D}=−0.650 (c=0.154, CHCl₃)

### HPLC Method for ee Determination:

Chiralpak-OD-H column, 25 cm*4.6 mm, 95% n-heptane+5% 2-propanol with 0.1% trifluroacetic acid, flow 0.7 ml/min, 30° C., 0.001 ml injection volume, 210 nm. Retention times: (−)-acid 9.5 min, (+)-acid 11.5 min.

### Assignment of the Absolute Configuration

The absolute configuration of the title compound was assigned as (3S,4R) by comparison of the optical rotation and the retention time by HPLC analysis on a Chiralpak-OD-H column with the values of a sample of (−)-(3S,4R)-4-(4-fluoro-phenyl)-piperidine-1,3-dicarboxylic acid 1-tert-butyl ester which was derived from (−)-(3S,4R)-4-(4-fluoro-phenyl)-1-methyl-piperidine-3-carboxylic acid methyl ester (prepared as described in WO0129031) as follows:

A solution of (−)-(3S,4R)-4-(4-fluoro-phenyl)-1-methyl-piperidine-3-carboxylic acid methyl ester (575 mg, 2.29 mmol) and 1-chloroethyl chloroformate (393 mg, 2.75 mmol) in 5 ml 1,2-dichloroethane was heated at reflux for 4 h. After cooling to room temperature and evaporation of the solvent in vacuo the residue was dissolved in 5 ml methanol. The solution was heated at reflux for 1 h, followed by cooling to room temperature and concentration in vacuo. The residue was dissolved in 11.5 ml of a 2 M aqueous solution of hydrochloric acid and heated at reflux over night. After cooling the reaction mixture to 0° C. on an ice-water bath were added consecutively 2.8 ml of a 32% aqueous solution of sodium hydroxide and a solution of di-tert-butyl dicarbonate (1.00 g, 4.58 mmol) in 15 ml 1,4-dioxane. The ice-water bath was removed after completed addition and stirring was continued at room temperature for 4 h. The pH of the reaction mixture was adjusted to 8 by the addition of 1 M aqueous sodium hydroxide solution. Washing with two portions of tert-butyl methyl ether was followed by back-extraction of the combined organic layers with 1 M aqueous sodium hydroxide solution. The combined aqueous layers were cooled to 0° C., acidified to pH 1 with ice-cold 4 M aqueous hydrochloric acid solution and extracted with three portions of ethyl acetate. The combined organic layers were washed with brine, dried over sodium sulfate and concentrated in vacuo to give (−)-(3S,4R)-4-(4-fluoro-phenyl)-piperidine-1,3-dicarboxylic acid 1-tert-butyl ester (590 mg, 80%) with 93.8% ee.

MS m/e (%): 322 (M−H⁺, 100).

[α]_{D}=−0.867 (c=0.462, CHCl₃)

1. A process for the preparation of enantiomerically enriched cyclic β-arylcarboxylic acid derivatives of formula 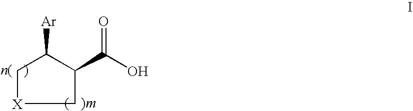
wherein
X is —C(R)(R′)—, —N(R″)—, —O—, —S(O)ₒ—, C(O)N(R″), —N(R″)C(O)— or —C(O)—;
R and R′ are each independently hydrogen, C₁₋₇-alkyl, C₁₋₇-alkyl substituted by halogen, C₁₋₇-alkoxy, hydroxy or —(CH₂)ₚ—Ar;
R″ is hydrogen, C₁₋₇-alkyl, C₁₋₇-alkyl substituted by halogen, —S(O)ₒ—C₁₋₇-alkyl, —S(O)ₒ—Ar, —S(O)ₙ—NRR′, —(CH₂)ₚ—Ar, —C(O)—C₁₋₇-alkyl, —C(O)—Ar, —C(O)—NRR′ or —C(O)O—C₁₋₇-alkyl;
Ar is aryl¹ or heteroaryl¹;
n is 0, 1, 2 or 3;
m is 0, 1, 2 or 3;
o is 0, 1 or 2; and
p is 0, 1, or 2;
or a pharmaceutically acceptable salt thereof
comprising catalytic homogeneous enantioselective hydrogenation of a compound of formula (II) 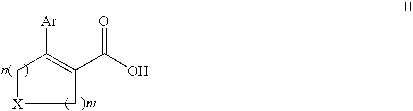
in the presence of a catalyst comprising
Ru(Z)₂D or XVII
[Ru(Z)₂₋ₚ(D)(L¹)ₘ](B)ₚ XVIII
wherein Z represents halogen or the group A-COO,
A represents lower alkyl, aryl², halogenated lower alkyl or halogenated aryl²,
D represents a chiral diphosphine ligand,
B represents a non-coordinating anion,
L¹ represents a neutral ligand,
p represents the numbers 1 and 2,
the ligands can be the same or different, and
m represents the numbers 1, 2 or 3.

2. The process of claim 1, wherein Z is CH₃COO, CF₃COO or a halogenide.

3. The process of claim 1, wherein the chiral diphosphine ligand is selected from the group consisting of 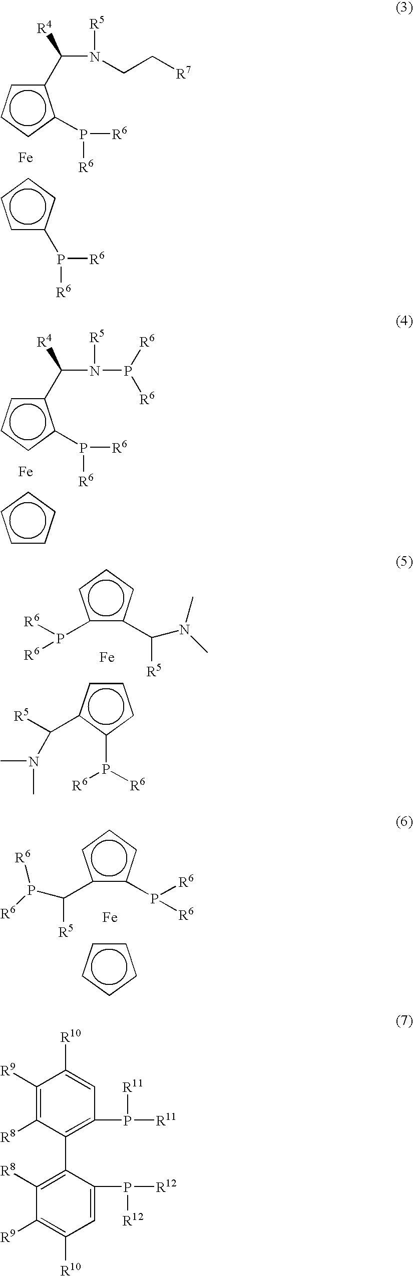 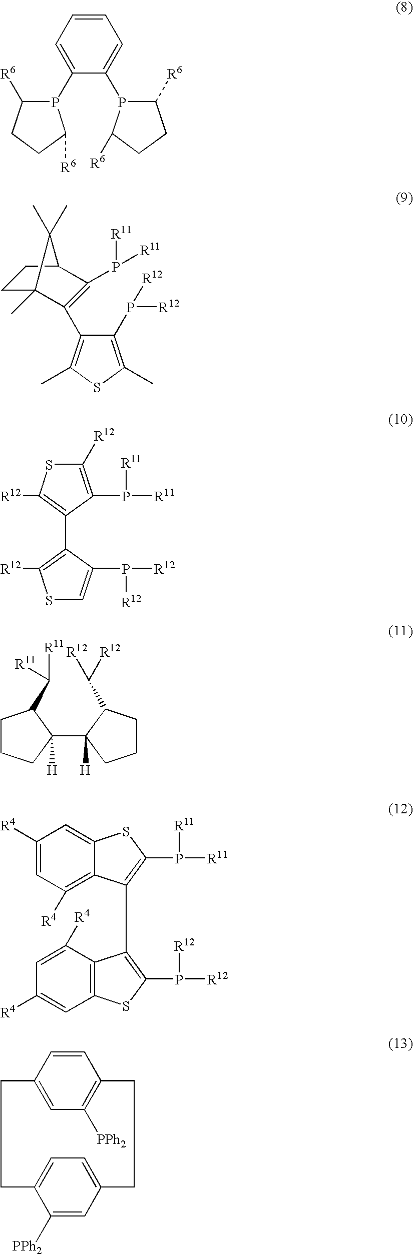 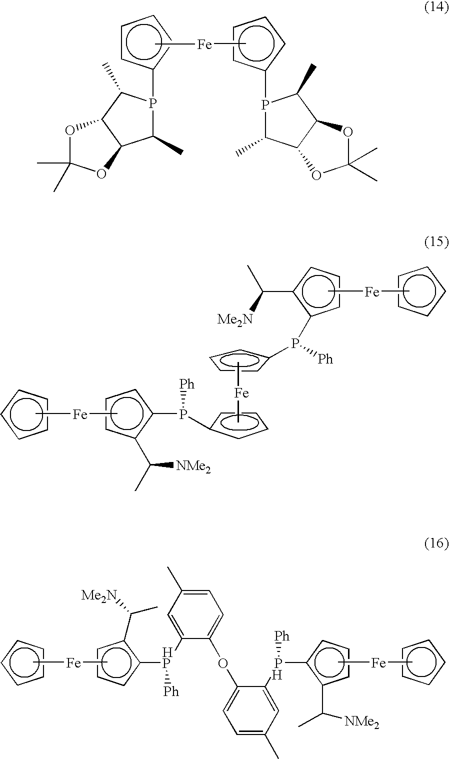
and
wherein
R⁴ is lower-alkyl;
R⁵ is lower-alkyl;
R⁶ is independently aryl², heteroaryl², cycloalkyl or lower-alkyl;
R⁷ is N(lower-alkyl)₂ or piperidinyl;
R⁸ is lower-alkyl, lower-alkoxy, hydroxy or lower-alkyl-C(O)O—;
R⁹ and R¹⁰ are each independently hydrogen, lower-alkyl, lower-alkoxy or di(lower-alkyl)amino;
or
R⁸ and R⁹ which are attached to the same phenyl group, or R⁹ and R¹⁰ which are attached to the same phenyl group, or both R⁸, taken together, are —X—(CH₂)ₙ—Y—, wherein X is —O— or —C(O)O—,
Y is —O— or —N(lower-alkyl)- and n is an integer from 1 to 6, or a CF₂ group; or
R⁸ and R⁹, or R⁹ and R¹⁰, together with the carbon atoms to which they are attached, form a naphthyl, tetrahydronaphthyl or dibenzofuran ring;
R¹¹ and R¹² are each independently lower alkyl, cycloalkyl, phenyl, napthyl or heteroaryl, substituted with 0 to 7 substituents independently selected from the group consisting of lower-alkyl, lower-alkoxy, di(lower-alkyl)amino, morpholino, phenyl and tri(lower-alkyl)silyl.

4. The process of claim 3, wherein D represents a chiral diphosphine ligand selected from the group consisting of formula (7), (9), (10) or (12).

5. The process of claim 4, wherein the chiral diphosphine is selected from the group consisting of (R) and (S)-enantiomers of MeOBIPHEP, BIPHEMP, TMBTP, 2-Naphthyl)-MeOBIPHEP, (6-MeO-2-Naphthyl)-MeOBIPHEP, 2-(Thienyl)-MeOBIPHEP, 3,5-tBu-MeOBIPHEP, PHANEPHOS, BICP, TriMeOBIPHEP, (R,R,S,S)-Mandyphos, BnOBIPHEP, BenzoylBIPHEP, pTol-BIPHEMP, tButylCOOBIPHEP, iPrOBIPHEP, p-Phenyl-MeOBIPHEP, pAn-MeOBIPHEP, pTol-MeOBIPHEP, 3,5-Xyl-MeOBIPHEP, 3,5-Xyl-BIPHEMP, BINAP and 2-Furyl-MeOBIPHEP, 3,5-Xyl-4-MeO-MeOBIPHEP, 2-Furyl-MeOBIPHEP, and BITIANP.

6. The process of claim 5, wherein the chiral diphosphine is (S)-(6-MeO-2-Naphthyl)-MeOBIPHEP, 3,5-Xyl-4-MeO-MeOBIPHEP, (S)-2-Furyl-MeOBIPHEP or BITIANP.

7. The process of claim 1, wherein the catalyst is selected from the group consisting of (R) and (S) enantiomers of [Ru(CH₃COO⁻)₂(TMBTP)], [Ru(CF₃COO⁻)₂(TMBTP)], [Ru(CH₃COO⁻)₂(2-naphthyl)-MeOBIPHEP)], [Ru(CF₃COO⁻)₂(2-naphthyl)-MeOBIPHEP)], [Ru(CH₃COO⁻)₂(6-MeO-2-naphthyl)-MeOBIPHEP)] and [Ru(CF₃COO⁻)₂(6-MeO-2-naphthyl)-MeOBIPHEP)].

8. The process of claim 1, wherein the catalytic hydrogenation is carried out at a pressure of 1 to 150 bar.

9. The process of claim 8, wherein the catalytic hydrogenation is carried out at a pressure of 10 to 100 bar.

10. The process of claim 1, wherein the catalytic hydrogenation is carried out at a temperature of 10 to 100° C.

11. The process of claim 10, wherein the catalytic hydrogenation is carried out at a temperature of 20 to 80° C.

12. The process claim 1, wherein the catalytic hydrogenation is carried out in the presence of a base.

13. The process of claim 12, wherein the base is selected from the group consisting of NEt₃, i-Pr₂NEt, i-Pr₂NH, C₆H₅CH₂NH₂, 1-phenyl-benzylamine, (R) or (S) ethylene diamine, tetramethylethylene diamine, NaOAc, NaOEt, NaOH and Bu₄NX, wherein X is F, Cl, Br or I.

14. The process of claim 13, wherein the base is NEt₃ or i-Pr₂Net.

15. The process of claim 1, wherein the catalytic hydrogenation is carried out in a solvent.

16. The process of claim 15, wherein the solvent is selected from the group consisting of an alcohol, hydrocarbon, chlorinated hydrocarbon, THF, water, and a mixture thereof.

17. The process of claim 16, wherein the solvent is methanol or ethanol.

18. The process of claim 15, wherein the concentration of solvents is 1-50 W %.

19. The process of claim 1, wherein the ratio of substrate/catalyst (s/C) is 5:30000.

20. The process of claim 1, wherein the compound of formula (I) is selected from the group consisting of
2-aryl/heteroaryl-cyclopentane carboxylic acids,
4-aryl/heteroaryl-2,5-dihydro-1H-pyrrolidine-3-carboxylic acids,
4-aryl/heteroaryl-tetrahydrofuran-3-carboxylic acids,
4-aryl/heteroaryl-tetrahydro-thiophene-3-carboxylic acids,
1,1-dioxo-4-aryl/heteroaryl-tetrahydro-1λ⁶-thiophene-3-carboxylic acids,
1-oxo-4-aryl/heteroaryl-tetrahydro-1λ⁴-thiophene-3-carboxylic acids,
2-aryl/heteroaryl-cyclohexane carboxylic acid,
4-aryl/heteroaryl-piperidine-3-carboxylic acids,
5-aryl/heteroaryl-piperidine-4-carboxylic acids, and
4-aryl/heteroaryl-tetrahydro-pyran-3-carboxylic acids or a pharmaceutically acceptable salt thereof.

21. The process of claim 1, wherein the compound of formula (I) is selected from the group consisting of
5-aryl/heteroaryl-tetrahydro-pyran-4-carboxylic acids,
4-aryl/heteroaryl-tetrahydro-thiopyran-3-carboxylic acids,
5-aryl/heteroaryl-tetrahydro-thiopyran-4-carboxylic acids,
1,1-dioxo-4-aryl/heteroaryl-hexahydro-1λ⁶-thiopyran-3-carboxylic acids,
1,1-dioxo-5-aryl/heteroaryl-hexahydro-1λ⁶-thiopyran-4-carboxylic acids,
1-oxo-4-aryl/heteroaryl-hexahydro-1λ⁴-thiopyran-3-carboxylic acids,
2-phenyl-cycloheptane carboxylic acid, and
2-phenyl-cyclooctane carboxylic acid or a pharmaceutically acceptable salt thereof.

22. A compound selected from the group consisting of
(+)-(3R,4R)-4-(4-fluoro-phenyl)-piperidine-1,3-dicarboxylic acid-1-tert-butyl ester,
(−)-(3S,4S)-4-(4-fluoro-phenyl)-piperidine-1,3-dicarboxylic acid 1-tert-butyl ester,
(−)-4-(4-(1H-indol-3-yl)-piperidine-1,3-dicarboxylic acid--tert-butyl ester,
(+)-4-(1H-indol-3-yl)-piperidine-1,3-dicarboxylic acid-1-tert-butyl ester,
(−)-4-o-tolyl-piperidine-1,3-dicarboxylic acid 1-tert-butyl ester,
(+)-4-o-tolyl-piperidine-1,3-dicarboxylic acid 1-tert-butyl ester,
(+)-4-(3-methoxy-phenyl)-piperidine-1,3-dicarboxylic acid-1-tert-butyl ester,
(+)-4-phenyl-piperidine-1,3-dicarboxylic acid-1-tert-butyl ester,
(−)-4-phenyl-piperidine-1,3-dicarboxylic acid-1-tert-butyl ester,
(+)-3-phenyl-piperidine-1,4-dicarboxylic acid 1-tert-butyl ester, and
(−)-3-phenyl-piperidine-1,4-dicarboxylic acid 1-tert-butyl ester.

23. A compound selected from the group consisting of
(−)-2-phenyl-cyclopentenecarboxylic acid,
(+)-2-phenyl-cyclopentenecarboxylic acid,
(+)-(3R,4R)-4-(phenyl)-pyrrolidine-1,3-dicarboxylic acid-1-tert-butyl ester,
(−)-(3S,4S)-4-(phenyl)-pyrrolidine-1,3-dicarboxylic acid-1-tert-butyl ester,
(−)-4-(4-chloro-phenyl)-pyrrolidine-1,3-dicarboxylic acid-1-tert-butyl ester,
(+)-4-(4-chloro-phenyl)-pyrrolidine-1,3-dicarboxylic acid-1-tert-butyl ester,
(+)-4-(3-fluoro-phenyl)-pyrrolidine-1,3-dicarboxylic acid-1-tert-butyl ester,
(−)-4-(3-fluoro-phenyl)-pyrrolidine-1,3-dicarboxylic acid-1-tert-butyl ester,
(3R,4R)-1-benzyl-4-phenyl-pyrrolidine-3-carboxylic acid,
(3RS,4RS)-1-benzyl-4-phenyl-pyrrolidine-3-carboxylic acid
(−)-2-phenyl-cyclooctanecarboxylic acid,
(+)-4-phenyl-tetrahydro-thiophene-3-carboxylic acid, and
(−)-4-phenyl-tetrahydro-thiophene-3-carboxylic acid.

24. A process of claim 1, wherein the starting compound of formula (II) 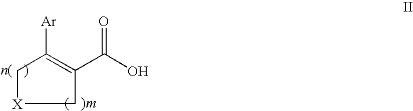
is selected from the group consisting of
2-aryl/heteroaryl-cyclopent-1-ene carboxylic acids,
4-aryl/heteroaryl-2,5-dihydro-1H-pyrrole-3-carboxylic acids,
4-aryl/heteroaryl-2,5-dihydro-furan-3-carboxylic acids,
4-aryl/heteroaryl-2,5-dihydro-thiophene-3-carboxylic acids,
1,1-dioxo-4-aryl-2,5-dihydro-1H-1λ⁶-thiophene-3-carboxylic acids,
2-aryl/heteroaryl-cyclohexyl-1-ene carboxylic acid,
4-aryl/heteroaryl-1,2,5,6-tetrahydro-pyridine-3-carboxylic acids,
5-aryl/heteroaryl-1,2,3,6-tetrahydro-pyridine-4-carboxylic acids, and
4-aryl/heteroaryl-5,6-dihydro-2H-pyran-3-carboxylic acids.

25. A process of claim 1, wherein the starting compound of formula (II) 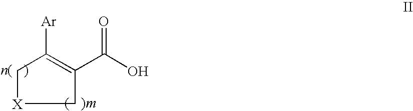
is selected from the group consisting of
5-aryl/heteroaryl-3,6-dihydro-2H-pyran-4-carboxylic acids,
4-aryl/heteroaryl-5,6-dihydro-2H-thiopyran-3-carboxylic acids,
5-aryl/heteroaryl-3,6-dihydro-2H-thiopyran-4-carboxylic acids,
1,1-dioxo-4-aryl/heteroaryl-1,2,5,6-tetrahydro-1λ⁶-thiopyran-3-carboxylic acids,
1,1-dioxo-5-aryl/heteroaryl-1,2,3,6-tetrahydro-1λ⁶-thiopyran-4-carboxylic acids,
1-oxo-4-aryl/heteroaryl-1,2,5,6-tetrahydro-1λ⁴-thiopyran-3-carboxylic acids,
1-oxo-4-aryl/heteroaryl-2,5-dihydro-1H-1λ⁴-thiophene-3-carboxylic acids,
2-phenyl-cyclohept-1-enecarboxylic acid and
2-phenyl-cyclooct-1-enecarboxylic acid.

26. A process for the preparation of enantiomerically enriched cyclic β-arylcarboxylic acid derivatives of formula 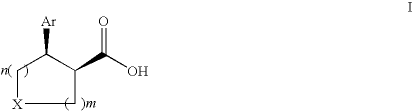
wherein
X is —C(R)(R′)—, —N(R″)—, —O—, —S(O)ₒ—, C(O)N(R″), —N(R″)C(O)— or —C(O)—;
R and R′ are each independently hydrogen, C₁₋₇-alkyl, C₁₋₇-alkyl substituted by halogen, C₁₋₇-alkoxy, hydroxy or —(CH₂)ₚ—Ar;
R″ is hydrogen, C₁₋₇-alkyl, C₁₋₇-alkyl substituted by halogen, —S(O)ₒ—C₁₋₇-alkyl, —S(O)ₒ—Ar, —S(O)O—NRR′, —(CH₂)ₚ—Ar, —C(O)—C₁₋₇-alkyl, —C(O)—Ar, —C(O)—NRR′ or —C(O)O—C₁₋₇-alkyl;
Ar is aryl¹ or heteroaryl¹;
n is 0, 1, 2 or 3;
m is 0, 1, 2 or 3;
o is 0, 1 or 2;
p is 0, 1, or 2;
or a pharmaceutically acceptable salt thereof
comprising catalytic homogeneous enantioselective hydrogenation of a compound of formula (II) 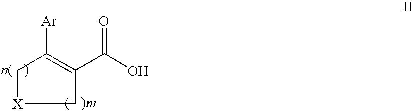
in the presence of a catalyst comprising
[Rh(chiral diphosphine)LX] or [Rh(chiral diphosphine)L]⁺A⁻
wherein X is Cl⁻, Br⁻ or I⁻,
L is a neutral ligand, selected from the group consisting of ethylene, propylene, cyclooctene, 1,3-hexadiene, norbornadiene, 1,5-cyclooctadiene, benzene, hexamethylbenzene, 1,3,5-trimethylbenzene, p-cymene, tetrahydrofuran, dimethylformamide, acetonitrile, benzonitrile, acetone and methanol,
A is an anion of an oxyacid or a complex acid selected from the group consisting of ClO₄, PF₆, BR₄, wherein R is halogen or aryl, SbF₆ and AsF₆.

27. The process of claim 26, wherein the chiral diphosphine ligand is selected from the group consisting of 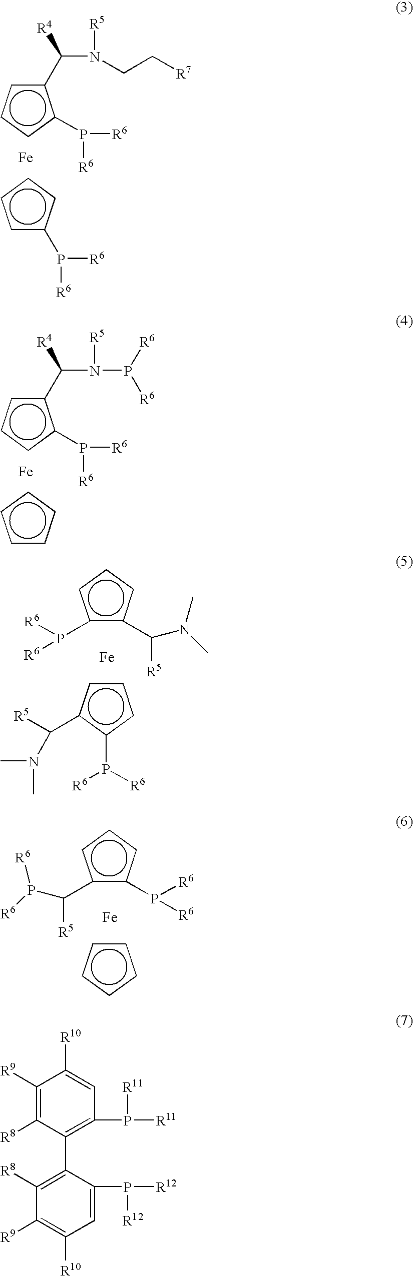 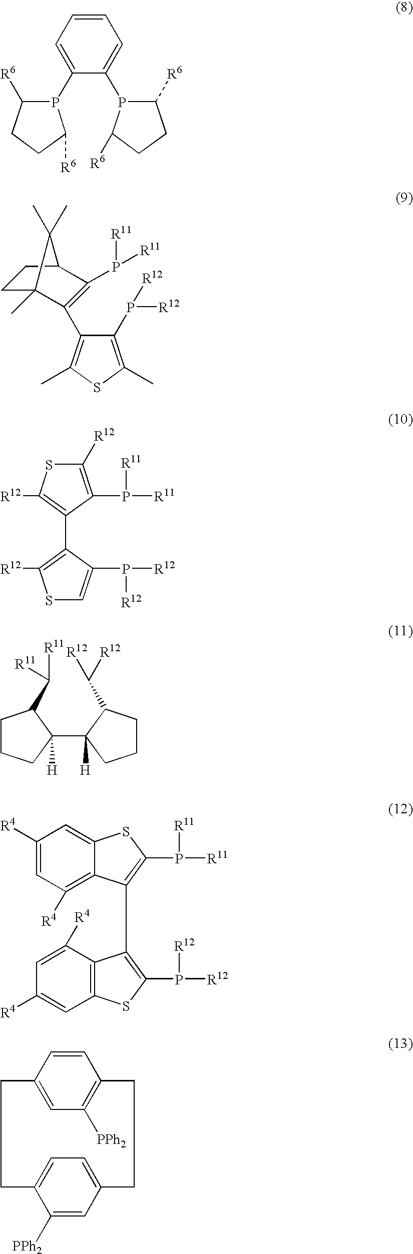 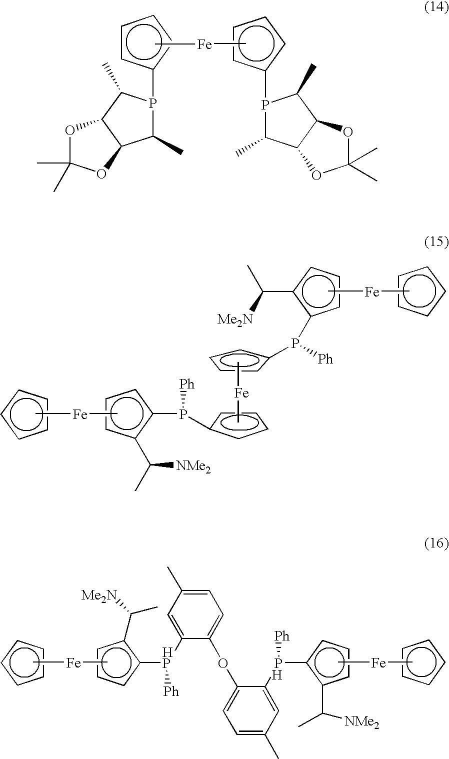
and
wherein
R⁴ is lower-alkyl;
R⁵ is lower-alkyl;
R⁶ independently is aryl², heteroaryl², cycloalkyl or lower-alkyl;
R⁷ is N(lower-alkyl)₂ or piperidinyl;
R⁸ is lower-alkyl, lower-alkoxy, hydroxy or lower-alkyl-C(O)O—;
R⁹ and R¹⁰ are each independently hydrogen, lower-alkyl, lower-alkoxy or di(lower-alkyl)amino; or
R⁸ and R⁹ which are attached to the same phenyl group, or R⁹ and R¹⁰ which are attached to the same phenyl group, or both R⁸, taken together, are —X—(CH₂)ₙ—Y—, wherein X is —O— or —C(O)O—, Y is —O— or —N(lower-alkyl)- and n is an integer from 1 to 6, or a CF₂ group; or
R⁸ and R⁹, or R⁹ and R¹⁰, together with the carbon atoms to which they are attached, form a naphthyl, tetrahydronaphthyl or dibenzofuran ring;
R¹¹ and R¹² are each independently lower alkyl, cycloalkyl, phenyl, napthyl or heteroaryl, substituted with 0 to 7 substituents independently selected from the group consisting of lower-alkyl, lower-alkoxy, di(lower-alkyl)amino, morpholino, phenyl and tri(lower-alkyl)silyl.

28. The process of claim 27, wherein D represents a chiral diphosphine ligand selected from the group consisting of formula (7), (9), (10) or (12).

29. The process of claim 28, wherein the chiral diphosphine is selected from the group consisting of (R) and (S)-enantiomers of MeOBIPHEP, BIPHEMP, TMBTP, 2-Naphthyl)-MeOBIPHEP, (6-MeO-2-Naphthyl)-MeOBIPHEP, 2-(Thienyl)-MeOBIPHEP, 3,5-tBu-MeOBIPHEP, PHANEPHOS, BICP, TriMeOBIPHEP, (R,R,S,S)-Mandyphos, BnOBIPHEP, BenzoylBIPHEP, pTol-BIPHEMP, tButylCOOBIPHEP, iPrOBIPHEP, p-Phenyl-MeOBIPHEP, pAn-MeOBIPHEP, pTol-MeOBIPHEP, 3,5-Xyl-MeOBIPHEP, 3,5-Xyl-BIPHEMP, BINAP and 2-Furyl-MeOBIPHEP, 3,5-Xyl-4-MeO-MeOBIPHEP, 2-Furyl-MeOBIPHEP, and BITIANP.

30. The process of claim 29, wherein the chiral diphosphine is (S)-(6-MeO-2-Naphthyl)-MeOBIPHEP, 3,5-Xyl-4-MeO-MeOBIPHEP, (S)-2-Furyl-MeOBIPHEP or BITIANP.

31. The process of claim 26, wherein the catalytic hydrogenation is carried out at a pressure of 1 to 150 bar.

32. The process of claim 31, wherein the catalytic hydrogenation is carried out at a pressure of 10 to 100 bar.

33. The process of claim 26, wherein the catalytic hydrogenation is carried out at a temperature of 10 to 100° C.

34. The process of claim 33, wherein the catalytic hydrogenation is carried out at a temperature of 20 to 80° C.

35. The process claim 26, wherein the catalytic hydrogenation is carried out in the presence of a base.

36. The process of claim 35, wherein the base is selected from the group consisting of NEt₃, i-Pr₂NEt, i-Pr₂NH, C₆H₅CH₂NH₂, 1-phenyl-benzylamine, (R) or (S) ethylene diamine, tetramethylethylene diamine, NaOAc, NaOEt, NaOH and Bu₄NX, wherein X is F, Cl, Br or I.

37. The process of claim 36, wherein the base is NEt₃ or i-Pr₂Net.

38. The process of claim 26, wherein the catalytic hydrogenation is carried out in a solvent.

39. The process of claim 38, wherein the solvent is selected from the group consisting of alkanols, benzene, toluene, trifluoro toluene, dichloromethane, dichlororethane, ethylene glycole, DMF, DMA, N-methylpyrrolidinone, acetonitrile, DMSO, and a mixture thereof.

40. The process of claim 38, wherein the concentration of solvents is 1-50 W %.

41. The process of claim 26, wherein the ratio of substrate/catalyst (s/C) is 5:30000.
